(19) United States
(12) Patent Application Publication
(10) Pub. No.: US 2006/0246484 A1
(43) Pub. Date: Nov. 2, 2006

(54) Identification of gene expression by heart failure etiology

(76) Inventors: Joshua M. Hare, Baltimore, MD (US); Michelle M. Kittleson, Los Angeles, CA (US)

Correspondence Address: COHEN, PONTANI, LIEBERMAN & PAVANE, 551 FIFTH AVENUE, SUITE 1210, NEW YORK, NY, 10176, US

(21) Appl. No.: 11/373,812

(22) Filed: Mar. 10, 2006

Related U.S. Application Data
(60) Provisional application No. 60/660,370, filed on Mar. 10, 2005.

(51) Int. Cl. C12Q 1/68; G06F 19/00

(52) U.S. Cl. 435/6; 702/20

(57) ABSTRACT

Differential gene expression profiles identifying heart failure etiology and the use thereof are disclosed.

### RELATED APPLICATIONS

This application claims priority from U.S. Provisional Patent Application Ser. No. 60/660,370 which was filed on Mar. 10, 2005, content of which is incorporated by reference in its entirety.

### BACKGROUND OF THE INVENTION

1. Field of Invention

The present invention relates to a gene expression profile, which provides information on heart failure etiology.

2. Related Art

Dilated cardiomyopathy is a common cause of congestive heart failure, the leading cause of cardiovascular morbidity and mortality in the United States (27). Dilated cardiomyopathy can be initiated by a variety of factors, such as ischemia, pressure or volume overload, myocardial inflammation or infiltration, and inherited mutations (14). A prevailing hypothesis is that, despite the varied inciting mechanisms that initiate the heart failure syndrome, there is a final common pathway that drives heart failure progression (47). Because of this, there is limited research into specific molecular events that are unique to the underlying process. This issue is especially relevant in the two major forms of dilated cardiomyopathy, nonischemic (NICM) and ischemic (ICM), While NICM and ICM have similar presentations (26), they are characterized by different pathophysiology, prognosis, and response to therapy (19; 21; 23; 24; 32; 42), and understanding their different pathophysiologic mechanisms is essential in guiding future therapies.

The emergence of microarray technology to simultaneously assess mRNA levels of tens of thousands of genes offers a novel approach to compare and contrast the myocardial transcriptome of NICM and ICM. Although previous studies have examined changes in gene expression in failing versus nonfailing (NF) hearts (2; 5; 44; 45; 51), they have focused only on NICM. The goal of this study was to simultaneously examine the differences in transcriptomes between either NICM or ICM and normal hearts to establish a set of shared and unique genes differentially expressed in the two major causes of heart failure. The present approach is distinct, but complementary, to our previous study (33) in which we used the method of nearest shrunken centroids (46) to determine a clinical prediction algorithm (i.e. a gene expression-based biomarker) that differentiated between NICM and ICM. The current analysis offers insight into both disease-specific pathogenesis and therapeutics. Furthermore, an understanding of the distinctions with potential pathophysiologic underpinnings between these two conditions supports and complements ongoing biomarker development efforts to differentiate heart failure of different etiologies (33).

Over the past two decades, there have been remarkable advances in medical and surgical therapies designed to improve the symptoms and survival of patients with heart failure, including angiotensin-converting enzyme (ACE) inhibitors, (62-64) beta-blockers, (65-58) aldosterone antagonists, (69-70) angiotensin-receptor blockers, (71-73) cardiac resynchronization therapy, (74-76) implantable defibrillators, (77-79) and ventricular assist devices.(80)

However, it is still not clear which patients will benefit most from which therapies, and a better understanding of the differences in response to therapy is essential because there are an increasing number of interventions that may be costly, such as implantable cardiac defibrillators; (81) risky, such as ventricular assist devices; (80) or scarce, such as donor hearts for cardiac transplantation.(82)

Thus, it is essential to determine if gene expression profiling through molecular signature analysis can distinguish between patients at different disease stages. One relevant disease stage is end-stage patients with and without left ventricular assist devices (LVADs). Patients with end-stage cardiomyopathy who are listed for cardiac transplantation all exhibit advanced heart failure. However, those who receive an LVAD prior to transplantation are a unique subset: patients who experience circulatory collapse before a heart becomes available and who would die if they did not receive mechanical circulatory support as a bridge to transplantation. Thus, these two types of end-stage cardiomyopathy patients form opposite ends of the clinical spectrum of advanced heart failure.

In this study, we have also shown that molecular signature analysis can be used to distinguish end-stage cardiomyopathy patients by stage of disease. This work supports our central hypothesis, that gene expression molecular signatures can be associated with clinically relevant parameters in heart failure patients and that these profiles can be applied prospectively in a diagnostic fashion.

### SUMMARY OF THE INVENTION

Cardiomyopathy can be initiated by many factors, but the pathway from unique inciting mechanisms to the common endpoint of ventricular dilation and reduced cardiac output is unclear. We previously described a microarray-based prediction algorithm differentiating nonischemic (NICM) from ischemic (ICM) cardiomyopathy using nearest shrunken centroids. Accordingly, we tested the hypothesis that NICM and ICM would have both shared and distinct differentially expressed genes relative to normal hearts and compared gene expression of 21 NICM and 10 ICM cardiomyopathy samples with that of 6 nonfailing (NF) hearts using Affymetrix U133A GeneChips and Significance Analysis of Microarrays. Compared to NF, 257 genes were differentially expressed in NICM and 72 genes in ICM. Only 41 genes were shared between the two comparisons, mainly involved in cell growth and signal transduction. Those uniquely expressed in NICM were frequently involved in metabolism, and those in ICM more often had catalytic activity. Novel genes included angiotensin-converting enzyme 2 (ACE2), which was upregulated in NICM but not ICM, suggesting that ACE2 may offer differential therapeutic efficacy in NICM and ICM. In addition, a tumor necrosis factor (TNF) receptor was downregulated in both NICM and ICM, demonstrating the different signaling pathways involved in heart failure pathophysiology. These results offer novel insight into unique disease-specific gene expression that exists between end-stage cardiomyopathy of different etiologies. This analysis demonstrates that transcriptome analysis offers insight into pathogenesis-based therapies in heart failure management, and complements studies using expression-based profiling to diagnose heart failure of different etiologies.

The present invention provides a differential gene expression profile, comprising comparative gene expression levels resulting from gene expressions of a set of genes from patients having nonischemic cardiomyopathy compared to gene expressions of a set of corresponding genes from patients having nonfailing-hearts and a differential gene expression profile, comprising comparative gene expression levels resulting from gene expressions of a set of genes from patients having ischemic cardiomyopathy compared to gene expressions of a set of corresponding genes from patients having nonfailing-hearts.

The present invention also provides a gene expression profile for distinguishing between patients with left ventricular assist devices (LVADs) and without LVADs, comprising the genes listed in Table 6.

### DESCRIPTION OF THE DRAWINGS

FIG. 1. Percent of known genes in each functional category that were significantly regulated in both nonischemic (NICM) and ischemic (ICM) cardiomyopathy compared to nonfailing (NF) hearts (black bars), unique to NICM hearts (gray bars), unique to ICM hearts (white bars), and the representation of these functional categories on the array (striped bars). There is no correlation with the representation of genes on the array and distribution of genes in the comparisons. APO is apoptosis, BIN is binding, CAT is catalytic activity, CEL is cell adhesion, CGM is cell growth/maintenance, CYT is cytoskeleton, DEV is development, INF is inflammatory response, MET is metabolism, NUC is nucleus, SIG is signal transduction, and TRA is transcription.

FIG. 2. Hierarchical clustering of genes based on similarity in gene expression and relatedness of samples. Each row represents a gene and each column represents a sample. Sample prefixes “T” denotes end samples from patients at the time of cardiac transplantation without left ventricular assist devices (no-LVAD); “LC” denotes samples obtained from patients at the time of LVAD placement (pre-LVAD), and “N” denotes nonfailing samples. The suffix “i” denotes ischemic cardiomyopathy samples. The suffix “ni” denotes nonischemic cardiomyopathy samples. The color in each cell reflects the level of expression of the corresponding gene in the corresponding sample, relative to its mean level of expression in the entire set of samples. Expression levels greater than the mean are shaded in blue, and those below the mean are shaded in red. Circled samples denote the predominant etiology clusters and samples labeled with an arrow fall outside of their appropriate cluster. A. Nonfailing versus ischemic cardiomyopathy. The no- and pre-LVAD samples do not form distinct clusters. B. Nonfailing versus nonischemic cardiomyopathy. The no- and pre-LVAD samples form distinct clusters, as indicated.

FIG. 3. Independent assessment of gene expression levels. To validate selected microarray findings using a complementary methodology, we quantified transcript abundance of 16 genes using quantitative PCR. Fold change in expression in nonischemic (NICM) and ischemic (ICM) hearts compared with nonfailing (NF) hearts according to QPCR (black bars) and microarrays (gray bars). ACE2, angiotensin-converting enzyme 2; ATP1B3, ATPase, Na+/K+ transporting, beta 3 polypeptide; FACL3, acyl-CoA synthetase long-chain family member 3; HBA2, hemoglobin A2; LEPR, leptin receptor; LUM, lumican; MYH6, myosin heavy chain 6; NAP1L3, nucleosome assembly protein 1-like 3; NPR3, atrionatriuretic peptide receptor C; PHLDA1, pleckstrin homology-like domain family A member 1; RPS4Y, ribosomal protein S4, Y-linked; S100A8, S100 calcium binding protein A8; SERPINE1, serine (or cysteine) proteinase inhibitor, clade E, member 1; SLC39A8, solute carrier family 39, member 8; TNFRSF11B, tumor necrosis factor receptor superfamily member 11 b; TXNIP, thioredoxin interaction protein. *P<0.05 compared with NF hearts by Wilcoxon rank sum test. \P<0.05 by Significance Analysis of Microarrays.

FIG. 4. Boxplots of the coefficient of variation for the gene transcripts identified as differentially expressed in nonischemic (NICM) and ischemic (ICM) hearts. The coefficient of variation is the standard deviation divided by the mean, and thus is a measure of variability that is not affected by the magnitude of the mean.

FIG. 5. Hierarchical clustering of genes based on similarity in gene expression and relatedness of samples. All 288 genes that were differentially expressed in either the nonfailing-ischemic or nonfailing-nonischemic comparison are included. Each row represents a gene and each column represents a sample. Sample prefixes “T” denotes end samples from patients at the time of cardiac transplantation without left ventricular assist devices (LVADs); “LC” denotes samples obtained from patients at the time of LVAD placement (pre-LVAD), and “N” denotes nonfailing samples. The suffix “i” denotes ischemic cardiomyopathy samples. The suffix “ni” denotes nonischemic cardiomyopathy samples. The color in each cell reflects the level of expression of the corresponding gene in the corresponding sample, relative to its mean level of expression in the entire set of samples. Expression levels greater than the mean are shaded in blue, and those below the mean are shaded in red. Circled samples denote the predominant etiology clusters.

FIG. 6. Hierarchical clustering of genes based on similarity in gene expression and relatedness of samples. Each row represents a gene and each column represents a sample. Sample prefixes “T” denotes end samples from patients at the time of cardiac transplantation without left ventricular assist devices (LYADs); “LC” denotes samples obtained from patients at the time of LVAD placement (pre-LVAD), and “N” denotes nonfailing samples. The suffix “i” denotes ischemic cardiomyopathy samples. The suffix “ni” denotes nomschemic cardiomyopathy samples. A. Nonfailing versus ischemic cardiomyopathy using those genes identified as differentially expressed in the nonfailing-nonischemic comparison. The samples do not form distinct etiology clusters. B. Nonfailing versus nonischemic cardiomyopathy using only those genes identified as differentially expressed in the nonfailing-ischemic comparison. The samples do not form distinct etiology clusters.

FIG. 7. Separation of end-stage cardiomyopathy samples into the training set (used to identify the molecular signature), test set (used to assess the accuracy of the signature).

FIG. 8. Heat map and unsupervised clustering algorithm of the seven significant genes in the pre-LVAD versus no-LVAD gene expression molecular signature. Each row represents a gene and each column represents a sample. A red cell denotes a gene that is underexpressed relative to the average expression in all samples. A blue cell denotes an overexpressed gene. The no-LVAD (NLV) and pre-LVAD (LV) samples segregate into two dominant clusters.

### DETAILED DESCRIPTION OF THE PREFERRED EMBODIMENTS

Methods

Patient Population

The study sample comprised 31 end-stage cardiomyopathy and 6 nonfailing (NF) hearts. Myocardial tissue from end-stage cardiomyopathy patients was obtained at the time of left ventricular assist device (LVAD) placement or cardiac transplantation from two institutions: 1) Johns Hopkins Hospital in Baltimore, Md. (n=24 NICM and ICM samples and 6 NF samples) and 2) University of Minnesota in Minneapolis, Minn. (n=7 NICM samples). Samples from the latter institution were collected and prepared independently (11), and gene expression data files were kindly provided.

Discarded myocardial tissue from the left ventricular free wall or apex obtained during surgery was immediately frozen in liquid nitrogen and stored at −80° C. There is no evidence that differences in left ventricular sampling sites contribute to sample variability, and in our previous experience, sampling tissue from these two sites did not contribute to variability in gene expression (33). The dissecting pathologist selectively excluded areas of visible fibrosis from the portion stored for analysis. Because myocardial tissue obtained at LVAD placement and unused donor hearts are considered discarded tissue, we obtained an exemption from the Johns Hopkins Institution Review Board for sample collection and medical chart abstraction without written informed consent.

Sample Preparation

ICM was defined as evidence of myocardial infarction on histology of the explanted heart. In addition, all patients with ICM exhibited severe coronary artery disease (>75% stenosis of the left anterior descending artery and at least one other epicardial coronary artery) and/or a documented history of a myocardial infarction (3; 4). Nonischemic cardiomyopathy (NICM) patients had no history of myocardial infarction, revascularization. or coronary artery disease and had all been diagnosed with idiopathic cardiomyopathy.

Microarray Hybridization

Myocardial RNA was isolated from frozen biopsy samples using the Trizol reagent and Qiagen RNeasy columns. Double-stranded cDNA was synthesized from 5 pg RNA using the SuperScript Choice system (Invitrogen Corp. Carlsbad, Calif.). Each double-stranded cDNA was subsequently used as a template to make biotin-labeled cRNA and 15 pg of fragmented, biotin-labeled cRNA from each sample was hybridized to an Affymetrix U I 33A microarray (Affymetrix, Santa Clara, Calif.). Affymetrix chip processing was performed at the Hopkins Program for Genomic Applications core facility. The U133A microarray allows detection of 21,722 transcripts (15,713 full length transcripts, 4,534 non-expressed sequence tags (ESTs) and 1,475 ESTs). The quality of array hybridization was assessed by the 3′ to 5′ probe signal ratio of GAPDH and β-actin. Our samples had a ratio of 1-1.2, indicating acceptable RNA preparation.

Data Normalization

We used the robust multi-array analysis (RMA) algorithm (5; 6) to pre-process the Affymetrix probe set data into gene expression levels for all 37 samples (the 30 samples prepared at our institution as described above and the 7 samples prepared at an outside institution (2)). The gene expression data files are accessible through the NCBI Gene Expression Omnibus (GEO) database (accession numbers for series GSEI 869: http://www.ncbi.nim.nih.gov/geo/).

Validation

Levels of transcript normalized to GAPDH (a constitutively expressed gene) were compared between NICM and NF samples and between ICM and NF samples to confirm the up- or down-regulation of differentially regulated transcripts. RNA was available from 4 nonfailing, 5 ischemic, and 10 nonischemic samples for analysis. The RNA was treated with DNasel to remove contaminating genomic DNA and subsequently used to synthesize cDNA. Primers were designed using Primer Express 2.0 software. Each sample was run on a GeneAmp 7900 Sequence Detection System (PE Applied Biosystems) and analyzed using SDS software (Applied Biosystems). For each gene of interest, a standard curve was generated using serial dilutions of a control cDNA. The quantity of gene transcript in unknown samples was estimated using this standard curve, with GAPDH as a normalizer. SYBR green reagent (Applied Biosystems) served as a reporter throughout all experiments.

We identified differentially expressed genes in two comparisons: 1) NICM versus NF hearts and 2) ICM versus NF hearts. Statistically significant changes in gene expression were identified using Significance Analysis of Microarrays (SAM) (49). SAM identifies genes with statistically significant changes in expression by identifying a set of gene-specific statistics (similar to the t-test) and a corresponding false discovery rate (FDR; similar to a p-value adjusted for multiple comparisons). Using the “one class” option, we identified genes with a FDR of <5% (corresponding to a p value adjusted for multiple comparisons <0.05) and an absolute fold change of ≧2.0. This threshold has been used in other similar studies (44) and may maximize specificity (20). These differentially expressed genes were visualized by hierarchical clustering (1) and heat mapping (22) using Euclidean distance with complete linkage.

Using a tissue repository of myocardial samples obtained from end-stage cardiomyopathy patients before and after placement of a left ventricular assist device (LVAD), we used well-established techniques to identify a gene expression molecular signature that distinguished subjects before and after LVAD placement. The gene expression signature was validated by testing its predictive accuracy prospectively in an independent set of samples. These results suggest that a gene expression signature previously identified that distinguishes patients by etiology (83) is distinct from that which distinguishes cardiomyopathy patients by disease stage.

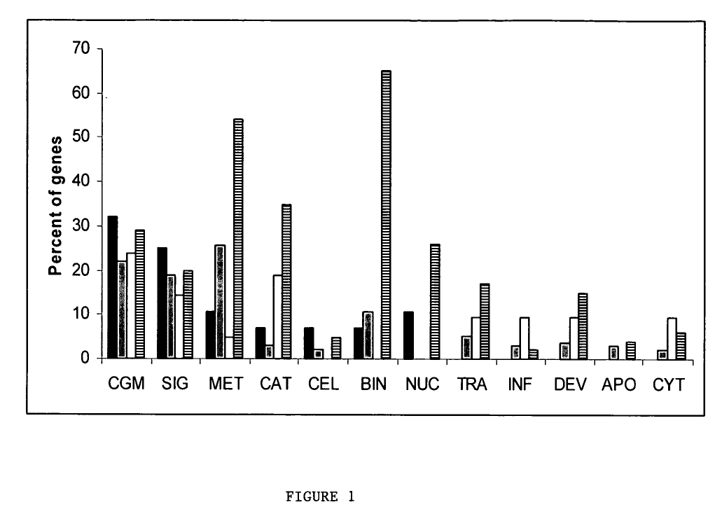

Myocardial tissue obtained from two separate institutions and from two sets of patients with advanced heart failure was examined: 1) 14 patients at the time of LVAD placement and 2) 11 patients who did not require an LVAD before transplantation (FIG. 1). With 12 samples, we used PAM to identify seven genes that distinguished patients with and without LVADs.

The expression signature included genes involved in transcription and signal transduction such as SP3 transcription factor (Table 1). When the profiles of these seven genes were applied to an independent set of 13 samples from two outside institutions, (62-65) all were correctly identified as with or without LVADs.

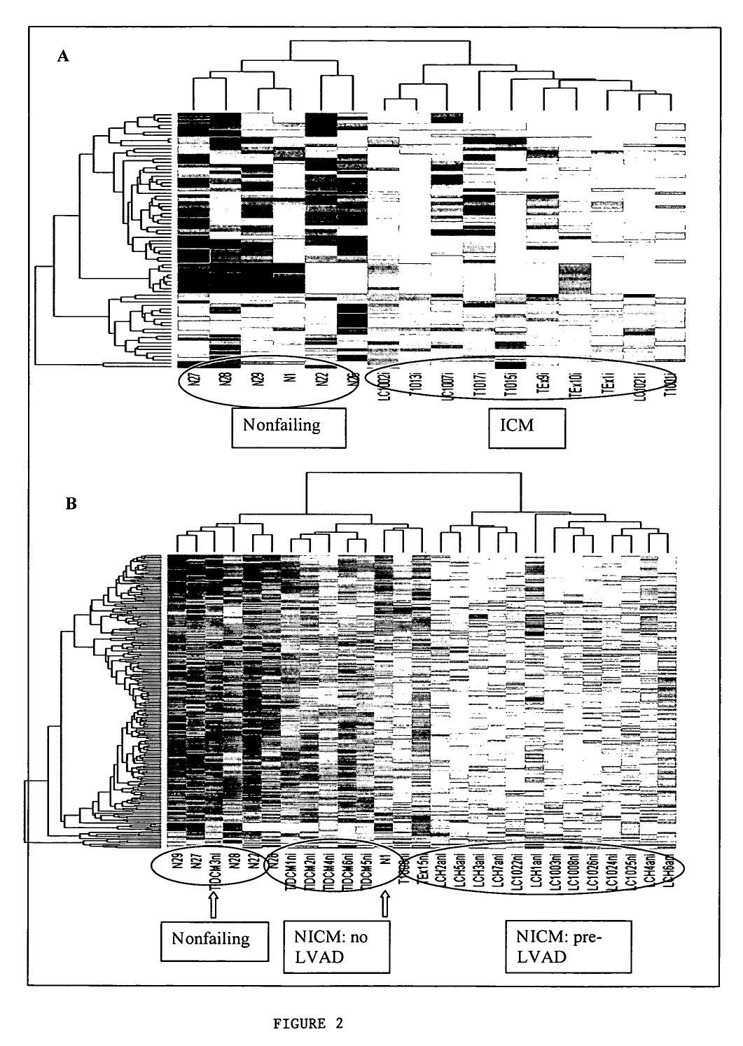

FIG. 2 illustrates the gene expression profiles of the 25 samples. Each row represents one of the seven genes, and each column is a patient sample. The dendrogram at the top is an unsupervised hierarchical clustering algorithm that divides samples into groups based on the similarity of the gene expression profiles. The two main clusters separate the LVAD patients (sample obtained at LVAD insertion) from those without LVADs. That gene expression profiling can differentiate clinical subsets of end-stage cardiomyopathy patients illustrates the sensitivity of this prediction tool. However, the gene expression prediction rule can also be applied successfully to samples from two outside institutions; illustrating the widespread applicability and generalizability of these techniques. Notably, this successful prediction was independent of the patients' age, gender, or medication history. This molecular signature represents a novel prognosis signature; even within the small spectrum of end-stage cardiomyopathy, a molecular signature is sensitive to patients with different disease severity.

Results

Clinical Specimens

Subjects with ischemic (n=10) or nonischemic (n=21) end-stage cardiomyopathy exhibited severely reduced ejection fraction, left ventricular dilation, elevated pulmonary arterial and wedge pressures, and reduced cardiac index (Table 1). Ischemic cardiomyopathy subjects were older, all male, more often on angiotension-convering enzyme inhibitors, and less often on intravenous inotropic therapy. Compared with no-LVAD patients, pre-LVAD patients had lower ejection fraction, higher pulmonary capillary wedge pressure, and lower cardiac index. The nonfailing hearts (n=6) were from unused cardiac transplant donors. The unused donor subjects were younger (median age 42 years with interquartile range 24-50 years), predominantly male, and echocardiographic and hemodynamic information and medications were not available.

Differential Gene Expression: NICM Versus NF and ICM Versus NF

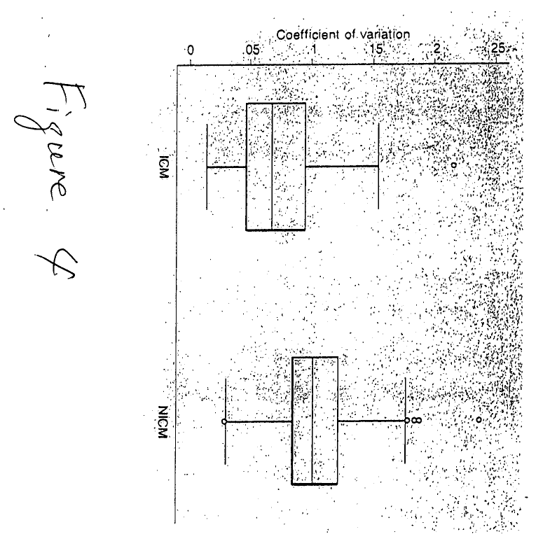

There were 257 genes differentially expressed between NICM and NF samples and 72 genes differentially expressed between ICM and NF samples with a false discovery rate of <5% and an absolute fold change of ≧2.0. Of the differentially expressed genes, only 41 were common to both NICM and NF and ICM and NF comparisons. As a measure of variability of gene expression, the coefficient of variation for these differentially expressed genes is depicted in FIG. 4. The coefficient of variation is low and comparable for both NICM and ICM.

Differentially Expressed Genes Common to Both NICM-NF and ICM-NF Comparisons

The majority of the 41 shared genes fell into functional classes of cell growth and maintenance and signal transduction (FIG. 1). Genes implicated in the fetal gene program induction were among those differentially expressed, including downregulation of alpha myosin heavy chain polypeptide 6 (36) and upregulation of atrionatriuretic peptide receptor C (18). In the cell growth and maintenance class, there were multiple probes corresponding to hemoglobin alpha and beta chains. There were also genes involved in signal transduction, including endothelin receptor type A and monocyte chemotactic protein 1. In addition, there were genes encoding components of the sarcomere (alpha myosin heavy chain noted above), the cytoskeleton (collagen type 21 alpha and ficolin), and the extracellular matrix (asporin). The majority of the genes were upregulated in NICM and ICM hearts compared with NF hearts, and for all 41 shared genes, fold changes were remarkably similar in direction and magnitude between NICM-NF and ICM-NF comparisons (Table 2).

Differentially Expressed Genes Unique to the NICM-NF Comparison

Of the 216 genes that were uniquely differentially expressed in NICM hearts, the majority fell into metabolism, cell growth and maintenance, signal transduction, and binding (FIG. 1 and Table 3 in Online Data Supplement). The genes involved in metabolism included angiotensin I-converting enzyme 2 (ACE2) and genes involved in fatty acid and cholesterol metabolism (acyl-CoA synthetase long-chain family member 3 and oxysterol binding protein-like 8). In cell growth and maintenance, upregulated genes included cyclin-dependent kinase inhibitor 1B and delta sleep inducing peptide, a vagal-potentiating peptide with influences on cardiac rhythm (39). Genes involved in signaling pathways were upregulated, included signal transducer and activator of transcription 1 and 4, members of the JAK/STAT signaling pathway, as well as receptors for leptin, growth hormone, transforming growth factor beta, and platelet-derived growth factor. Several genes implicated in inflammation and the immune response showed increased expression in NICM hearts, including interleukin 27, an MHC molecule, and a component of the complement pathway, H factor 1. There were also several genes related to cell adhesion, apoptosis, and development. All genes were upregulated in NICM hearts except one: a zinc transporter which was downreguled 2-fold.

Differentially Expressed Genes Unique to the ICM-NF Comparison

The 31 genes uniquely differentially expressed between NF and ICM hearts were predominantly in functional classes of cell growth and maintenance, catalytic activity, and signal transduction (FIG. 1 and Table 4). They also included genes implicated in the fetal gene program induction, including upregulation of natriuretic peptide precursor B, atrial natriuretic factor, and an embryonic atrial myosin light chain polypeptide (14).

Differentially Expressed Genes and Functional Categories

As shown in FIG. 1, the majority of genes on the array (over 50%) belonged to functional classes of binding and metabolism; a moderate number of genes (15-40%) were in the classes of catalytic activity, cell growth/maintenance, development, nucleus, signal transduction, and transcription; and few genes (less than 10%) belonged to classes of apoptosis, cell adhesion, cytoskeleton, and inflammatory response (the combined percentages total over 100% since genes can belong to more than one functional category). This pattern does not match that of our data (p<0.001 in a x² test). This suggests that the differences in functional categories identified were not solely a function of their representation on the microarray.

Clustering

The heat maps with clustering algorithms for the two comparisons, ICM-NF and NICM-NF, is shown in FIG. 2. The NF samples formed a distinct cluster from the ICM samples. For the NICM-NF comparison, there were two dominant clusters. One dominant cluster contained only NICM samples obtained from patients at the time of LVAD implantation (NICM/pre-LVAD). The other dominant cluster contained two subgroups: 1) predominantly NF samples and 2) the remaining portion of NICM samples, which were all obtained from patients who did not have an LVAD prior to cardiac transplantation (NICM/no-LVAD). Thus, there was a clear discrimination among the NICM samples of those obtained from 1) patients who required LVADs prior to cardiac transplantation and 2) patients who survived to cardiac transplantation without LVAD support.

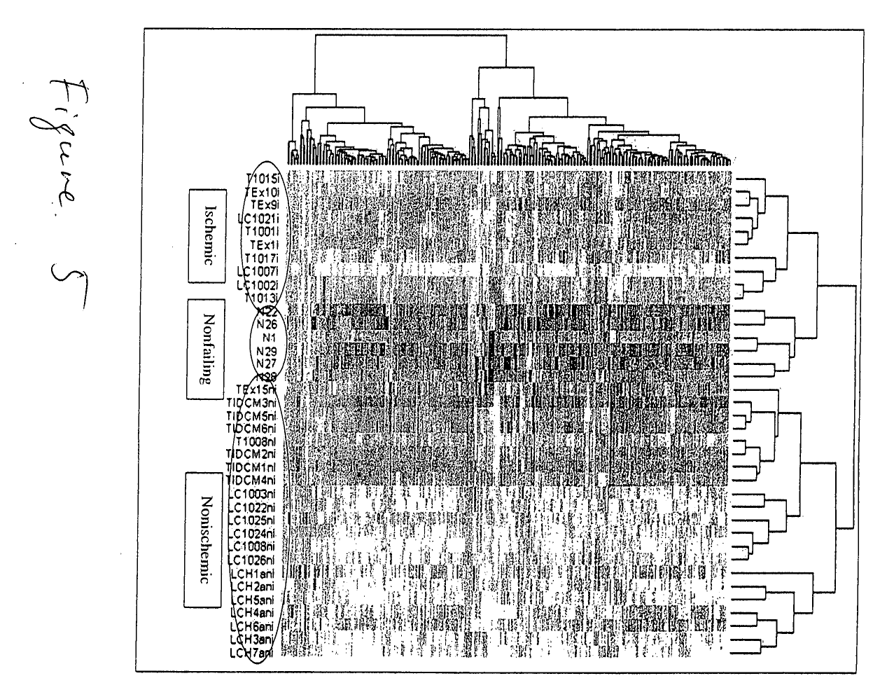

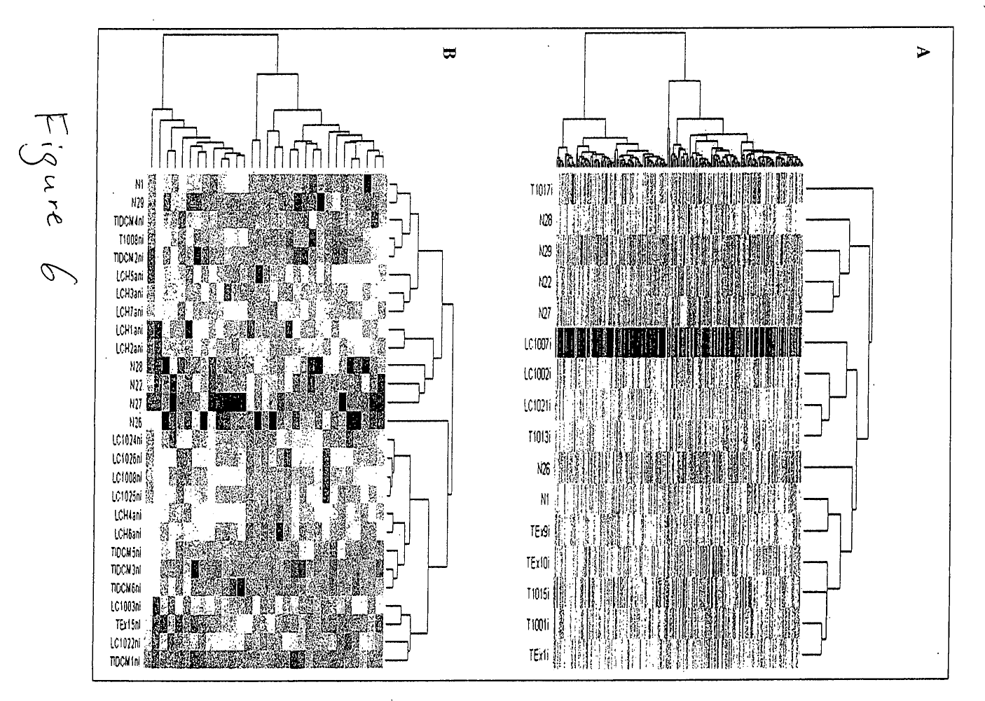

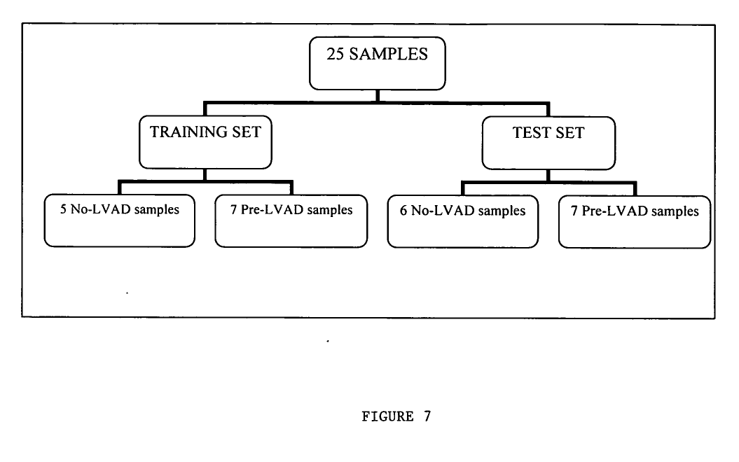

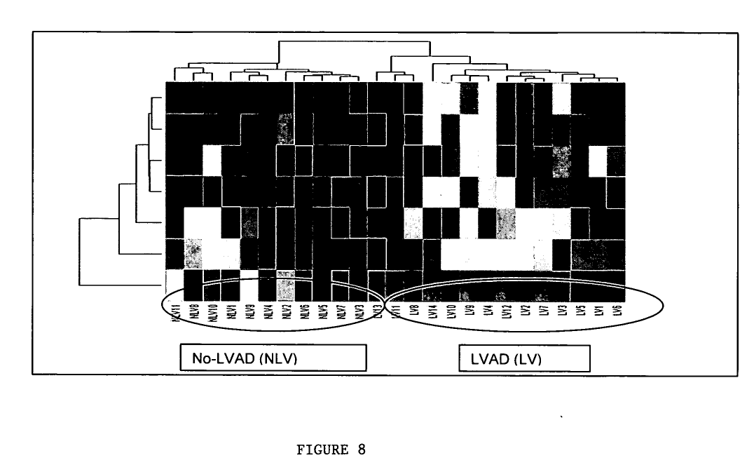

To determine the specificity of the profiles, we also created a heat map with clustering algorithm for all 288 genes that were identified as differentially expressed in at least one of the two comparisons (FIG. 5). Samples formed three distinct etiology clusters, NF, ICM, and NICM, but this was likely due to the presence of shared differentially expressed genes. To confirm the specificity of the differentially expressed genes, we performed two additional heat maps with clustering (FIGS. 6A and 6B): first, NF and ICM samples using only those genes identified as differentially expressed between NF and NICM samples, and second, NF and NICM samples using only those genes identified as differentially expressed between NF and ICM samples. If, as we assumed, the genes uniquely identified as differentially expressed in ICM relative to NF hearts were truly unique to the ICM-NF comparison, then a heat map of these genes in NICM and NF hearts should demonstrate no clustering by etiology, and vice versa for NICM genes in ICM hearts. This was the case: as expected, in both heat maps, the samples did not cluster by etiology, indicating that the unique differentially expressed genes were specific to the given comparison.

Validation

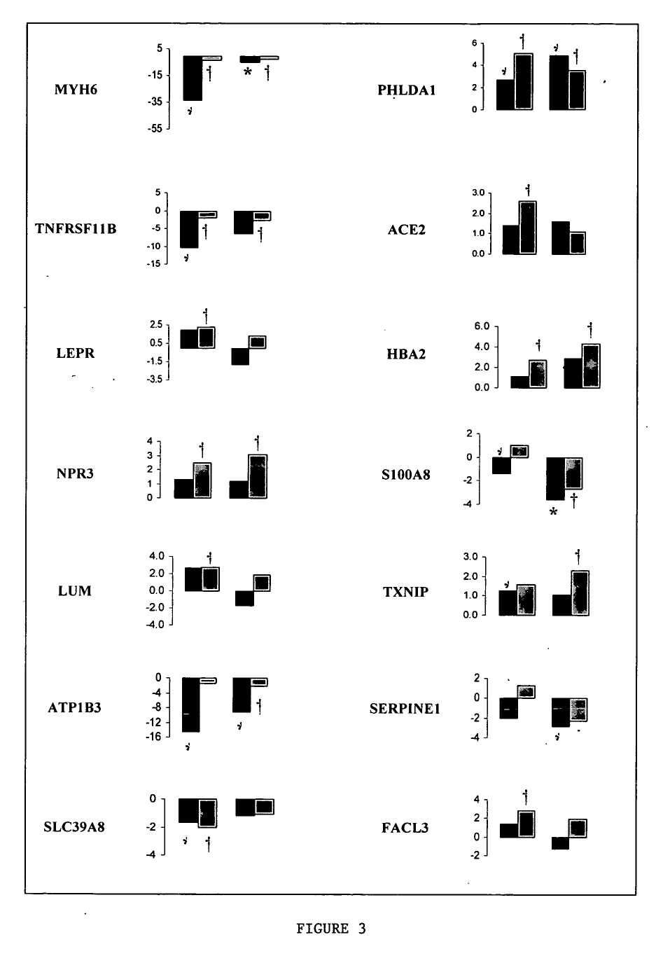

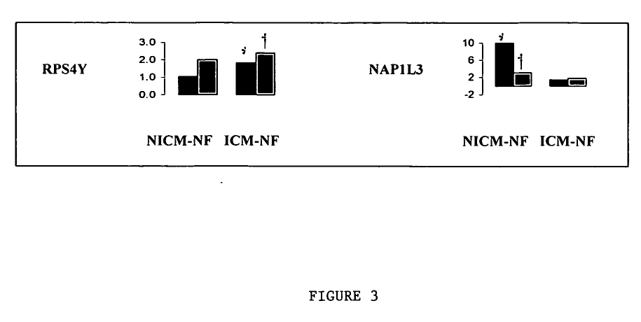

We selected 16 genes of potential biologic interest and validated the microarray findings in NICM, ICM, and NF hearts using QPCR. As shown in FIG. 3, QPCR confirmed 27 of the 32 microarray predictions with regard to fold change; 11 of these agreed completely in fold change and significance. Of the 5 that did not agree on fold change, 3 were nonsignificantly changed in both comparisons (the leptin receptor in ICM, serine proteinase inhibitor, lade E, member 1 in NICM, and the acyl-CoA synthetase long-chain family member 3 in ICM), leaving only 2 clear disagreements: S100 calcium binding protein A8 was significantly downregulated by QPCR but nonsignificantly upregulated by microarray and lumican was significantly upregulated in ICM by microarray and nonsignificantly downregulated by QPCR. Notably, of the 10 genes significantly expressed only in one comparison, NICM or ICM, relative to NF hearts, 17 of the 20 comparisons were confirmed by fold change and/or significance, again confirming the specificity of the uniquely identified genes.

Discussion

The principal finding of this investigation is that cardiomyopathies of different etiologies exhibit both shared and distinct changes in gene expression compared with nonfailing hearts. Remarkably, of the almost 22,000 transcripts present on the Affymetrix microarray platform, only a total of 288 genes are differentially expressed in NICM and ICM relative to NF hearts, and 41 of these genes are common to both comparisons with comparable fold changes. This suggests that there are both shared and distinct mechanisms that contribute to the development of heart failure of different etiologies, which supports the recent identification of gene expression-based diagnostic biomarker that differentiates between ischemic and nonischemic cardiomyopathy (33). In addition, a better understanding of these distinctions encourages ongoing efforts to develop cause-specific therapies specifically targeted at NICM and ICM (7).

These results complement our recent identification of a gene expression profile that differentiates between ischemic and nonischemic cardiomyopathy (33). In that analysis, we used Prediction Analysis of Microarrays (46) to identify and validate a 90-gene profile could differentiate between NICM and ICM. Unlike the current analysis, Prediction Analysis of Microarrays identifies the smallest number of genes that succinctly characterizes a class. These genes do not necessarily have biologic significance, since they are chosen based on the stability of their expression rather than a combination of magnitude and stability (46). This study demonstrated that gene expression profiles correlated with clinical parameters in heart failure patients and supported ongoing efforts to incorporate expression profiling-based biomarkers in determining prognosis and response to therapy in heart failure.

The current study has a distinctly different purpose, and uses different samples and statistical methods. Instead of identifying and validating a gene expression profile as a diagnostic biomarker, the current study focuses on novel gene discovery: identifying differentially expressed genes to better understand the similarities and differences between the two major forms of cardiomyopathy, ICM and NICM. In addition, because we were interested in the genesis of cardiomyopathy, we compared both ICM and NICM to NF hearts (the prior study did not involve NF hearts). Finally, in the current study, we used Significance Analysis of Microarrays (49) to identify differentially expressed genes, and validated our findings with qPCR, as opposed to using Prediction Analysis of Microarrays, and validating our findings by testing the gene expression prediction profile in an independent set of samples.

Thus, the two studies target two different goals of microarray analysis, using a pattern of gene expression as a biomarker versus examining gene expression for novel gene discovery (7; 15). These findings of the unique and shared genes expressed in NICM and ICM relative to NF hearts complements those of the prior study. Both demonstrate that unique gene expression exists in the two major forms of cardiomyopathy. On one hand, this allows a pattern of gene expression to function as a diagnostic biomarker. On the other hand, the unique patterns of gene expression can be further investigated to better define cause-specific therapies for heart failure. These two analyses are clearly not redundant, since they used different sets of samples, different statistical methods, and most importantly, had different purposes. Furthermore, given the complementary nature of the two analyses, it is not surprising that only four of the genes in the current study were observed in our prior identification of a gene expression profile that differentiated between ICM and NICM (33). The current analysis also focused on differential gene expression, and thus targeted different genes than one investigating prediction (46).

The current study is unique for a number of reasons. First, we have studied 37 samples, which is a large number relative to gene expression studies in cardiomyopathy to date (2; 3; 5; 10; 11; 25; 28; 44; 45; 51). There are no accepted means of calculating sample size and power in microarray experiments, but because our study examines a larger number of samples than prior studies, we have increased power to detect significant changes in gene expression. Furthermore, we have the added advantage of uniformity among samples: all NICM hearts were from individuals with idiopathic cardiomyopathy, and the clinical characteristics were reasonably similar within groups.

The second unique feature of this study is that we have not compared only failing and nonfailing hearts, as in many previous studies (2; 5; 45; 51), but extended this analysis to compare the differential gene expression of NICM and ICM relative to NF hearts. This offers further insight into the mechanisms involved in the development of heart failure of varying etiologies. The majority of genes are shared between NICM and ICM relative to NF hearts, and this is consistent with clinical experience: the presentations and standard treatment for cardiomyopathy of both etiologies is similar (27). However, despite similar presentations and therapies, NICM and ICM are distinct diseases; patients with ICM have decreased survival compared with their NICM counterparts (21; 24), and respond differently to therapies (19; 23; 32; 42). Thus, an understanding of the distinctions between the two conditions at the level of gene expression may guide future efforts to design etiology-based therapies.

The predominance of metabolism genes in NICM hearts suggests that the derangements involved in the genesis and maintenance of NICM may be metabolic in nature. This is supported by an early trial of beta-blockers in heart failure which demonstrated a greater mortality benefit in NICM than ICM (13). Beta-blockers improve myocardial efficiency by shifting myocardial metabolism from free fatty acids to glucose. The increase in fatty acid metabolism genes specifically in NICM in our analysis would explain why beta-blockers may be particularly beneficial in NICM. Furthermore, our results suggest that future etiology-specific therapies in NICM could target metabolic pathways, including those of fatty acid or cholesterol synthesis. One particularly relevant example is ranolazine. This investigational compound shifts myocardial cells from fatty acid to glucose metabolism and is currently being investigated as a treatment for myocardial ischemia (9). Based on our results, this drug could also be helpful in patients with NICM.

In ICM, on the other hand, our results suggest that abnormalities in catalytic activity may predominate, and an anti-ischemic protective effect of the specific catalytic enzymes indentified, serine proteinase inhibitors, has been previously observed in pigs subject to experimentally-induced myocardial ischemia (31). Given our results, it may be possible that such enzymes could also be beneficial in patients with ICM.

Our work agrees to an extent with the findings of a similar analysis of differential gene expression by Steenman et al. (44), in which pooled samples of NICM and ICM were compared to one NF sample, and 95 differentially expressed genes were identified between failing and nonfailing hearts. When compared to our list of 288 genes, we found 8 genes in common (Table 5). There are a number of reasons why our results differed from those of the prior study. The prior study had only one NF heart, and it was from a patient with cystic fibrosis. This heart is likely very different, not only in age, but also in hemodynamic parameters, from a heart from an unused cardiac transplant donor. In addition, we used different statistical algorithms for normalization and identification of differentially expressed genes. We normalized with RMA, which has been shown to offer better detection of differentially expressed genes than Affymetrix's default preprocessing algorithm (29). We identified differentially expressed genes with Significance Analysis of Microarrays, which has been validated in a number of studies (6; 41; 49; 50) and may be more accurate than other commonly used methods for identifying differentially expressed genes, such as t-tests (43). In addition, our analysis may have more external validity because we studied more samples (37 versus 7 patients) with individually hybridized, as opposed to pooled, data. Individual hybridization may be more accurate than pooling because it allows the estimation of the within-group variance for each gene (38).

Some of the genes shown to be differentially expressed in our study have been previously identified as differentially expressed in studies of NF versus NICM hearts, with remarkably similar fold changes between studies (Table 5). Commonly identified genes include those involved in the fetal gene program (14), including natriuretic peptide precursor B, atrial natriuretic factor, cardiac muscle myosin heavy chain, and atrial alkali myosin light chain. The majority of genes are upregulated in NICM and ICM hearts versus NF hearts, and this has also been noted in prior studies (2; 5; 44; 45; 51). This is likely due to biologic differences, since prior studies all used different methods to normalize data and identify differentially expressed genes. Furthermore, since the expression of many of these genes was confirmed with quantitative PCR in these prior studies, this offers indirect further confirmation of the validity of our differentially expressed genes. This highlights the critical point in microarray analysis used for gene discovery: the results should be considered hypothesis-generating and the gene expression should be confirmed with other quantitative techniques, such as quantitative PCR (15).

Through quantitative PCR, we confirmed the expression of 27 of the 32 comparisons with 16 genes of interest in heart failure. Of greatest interest are the novel genes from our analysis, including ACE2 and a member of the tumor necrosis factor receptor superfamily (TNFRSF11B, also known as osteoprotegerin). ACE2 is expressed predominantly in vascular endothelial cells of the heart and kidney, and ACE and ACE2 have different biochemical activities. Angiotensin I is converted to angiotensin I-9 (with nine amino acids) by ACE2 but is converted to angiotensin II, which has eight amino acids, by ACE. Whereas angiotensin II is a potent blood-vessel constrictor, angiotensin I-9 has no known effect on blood vessels but can be converted by ACE to a shorter peptide, angiotensin I-7, which is a blood-vessel dilator (4). Loss of ACE2 was associated with up-regulation of hypoxia-inducible genes, suggesting a role for ACE2 in mediating the response to cardiac ischemia (17). Furthermore, the upregulation of ACE2 is ischemic but not nonischemic cardiomyopathy cannot be ascribed to the increased prescription of ACE inhibitors in ischemic cardiomyopathy subjects because unlike ACE, ACE2 is insensitive to inhibition by ACE inhibitors (48). Thus, we now show that in subjects with end-stage cardiomyopathy, ACE2 is significantly upregulated in nonischemic but not ischemic cardiomyopathy, suggesting that increasing levels of ACE2 may be an adaptive response to nonischemic but not ischemic heart failure.

Another novel finding of interest is the significant downregulation of a member of the tumor necrosis factor receptor subfamily, TNFRSF11B in both NICM and ICM. Levels of tumor necrosis factor (TNF) have been shown to be upregulated in chronic heart failure (34) and increasing levels of TNF have been correlated with disease severity (40). However, in clinical trials, soluble TNF-alpha antagonists did not reduce mortality or heart failure hospitalizations (12; 37). One might speculate that this lack of benefit may relate somehow to the down-regulation of the TNF receptor in chronic heart failure.

The results of the unsupervised hierarchical clustering algorithm suggest that patients with NICM patients who do not undergo LVAD implantation resemble nonfailing hearts more than NICM patients who require an LVAD prior to cardiac transplantation. An examination of their baseline characteristics confirms this: NICM-LVAD patients are a sicker subset, with higher pulmonary capillary wedge pressure and increased need for intravenous inotropes, two known markers of poor prognosis in chronic heart failure patients (8; 16). While there are documented changes in gene expression between hearts before and after LVAD support (3; 10; 11; 25), there is no evidence that differential gene expression exists between end-stage cardiomyopathy samples obtained before LVAD placement and at the time of cardiac transplantation or between patients with different clinical presentations. Because this result was obtained with an unsupervised clustering algorithm, it is free of bias of predefined categories (35). While is it possible that the differences were due, in part, to the use of 7 NICM-LVAD samples from an outside institution, this is less likely based on our prior results with these samples, which indicated that the institution of origin did not contribute to variability in gene expression (33) and because the outside institution samples themselves did not form a distinct cluster. This unanticipated difference between end-stage NICM patients could offer insight into the differential gene expression of different stages of heart failure. This requires further study, and lends credence to the notion that gene expression can be correlated with clinically relevant parameters in heart failure patients to aid in determining prognosis and response to therapy.

Although the analysis of gene expression using oligonucleotide microarrays is a powerful technique, limitations warrant mention. Not all genes are represented on the Affymetrix U133A arrays used in this study, and therefore the knowledge that can be acquired from these experiments remains incomplete. In addition, a nonfailing, unused donor heart is not the same as a normal heart, because circumstances causing to a donor heart being ineligible for cardiac transplantation, such as infection or prolonged hypotension, can also affect gene expression. In fact, one study suggested that the differential gene expression identified between failing and nonfailing hearts may have been due to age and gender differences rather than differences in ventricular function (5). However, normal, age- and sex-matched hearts are impossible to obtain, and other researchers have used comparable unused donor hearts in their experiments (2; 5; 45; 51).

Another limitation of this study is that microarray analysis is essentially hypothesis generating. However, in the tradition of such studies in the microarray literature (2; 3; 5; 10; 11; 25; 30; 44; 45; 51), this is a hypothesis-generating analysis with biologic validation of select genes confirmed by QPCR. We have followed the practice of other studies in the field, and extended the analysis to include more samples with different etiologies of heart failure and a careful comparison with the results of prior studies (Table 5), which is unprecedented in the literature thus far. For this reason, we believe that these analyses, while mainly hypothesis-generating, do have significant value and should be made available to other individuals interested in microarray analysis of ischemic and nonischemic cardiomyopathy.

In conclusion, we offer a novel addition to the analysis of differential gene expression between failing and nonfailing hearts by providing new insight into the genetic pathways involved in the transition to cardiomyopathy of different etiologies. By comparing differential gene expression in nonischemic and ischemic cardiomyopathy relative to nonfailing hearts, we have shown that there are a number of common and unique genes involved in the development of heart failure of differing etiologies. This analysis will provide valuable hypothesis-generating insight into the pathophysiology of heart failure and offers a basis for future studies of cause-specific therapies in the complex management of heart failure patients.

TABLE 1
Clinical characteristics

| | Ischemic | | Nonischemic | |
|---|---|---|---|---|
| | No LVAD* | Pre-LVAD† | No LVAD* | Pre-LVAD* |
| | (n = 7) | (n = 3) | (n = 8) | (n = 13) |
| Age, y | 54 (49-60) | 60 (59-60) | 51 (48-53) | 46 (37-52)§ |
| Male | 100% | 100% | 86% | 62% |
| Ejection fraction, % | 20 (15-25) | 17.5 (10-25) | 17.5(7.5-27.5) | 15 (12.5-20) |
| LVIDd, cm | 6.8 (6.7-7.6) | 6.5 (6-7) | 8.4 (7.5-9.3) | 7.3 (6.8-8.1) |
| PCWP, mm Hg | 15 (12-23) | 30 (30-32) | 13.5 (13-14) | 27 (21-31)‡ |
| Cardiac index, L · min¹ · m² | 2.4 (2.3-2.4) | 1.4 (1.3-1.5)‡ | 2.4 (1.9-2.8) | 1.5 (1.3-1.6) |
| Beta antagonists | 71% | 67% | 38% | 36% |
| ACE inhibitors or ARBs | 100% | 100% | 88% | 55% |
| Diuretics | 100% | 100% | 100% | 64% |
| Inotropic therapy^{a} | 100% | 33% | 13% | 73%‡ |

Values are median (25^{th} and 75^{th} percentiles) *, median (range) †, or percentages.
ACE is angiotensin-converting enzyme,
ARB is angiotensin receptor blocker,
LVAD is left ventricular assist device;
LVIDd is left ventricular end-diastolic diameter,
PCWP is pulmonary capillary wedge pressure.
‡p < 0.05 for difference between no-LVAD and pre-LVAD groups.
§p < 0.05 for difference between ischemic and nonischemic cardiomyopathy.
^{a}Includes dopamine, dobutamine, and milrinone.

TABLE 2
Differentially expressed genes shared between the ischemic-cardiomyopathy-versus-nonfailing
heart and nonischemic-cardiomyopathy-versus-nonfailing-heart comparison

| | | ICM-NF | | NICM-NF | |
|---|---|---|---|---|---|
| | | Fold | | Fold | |
| Gene symbol | Gene name | change* | FDR | change* | FDR |
| Cell | | | | | |
| growth/maintenance | | | | | |
| HBA2 | hemoglobin, alpha 2 | 4.3 | 0.50 | 2.7 | 0.18 |
| HSAGL2 | human alpha-globin gene | 3.5 | 0.50 | 2.4 | 0.18 |
| HBB | hemoglobin, beta | 3.4 | 0.50 | 2.6 | 0.18 |
| HBA2 | hemoglobin, alpha 2 | 3.4 | 0.50 | 2.2 | 0.18 |
| HBA1 | hemoglobin, alpha 1 | 3.3 | 0.50 | 2.1 | 0.18 |
| AF059180 | mutant beta-globin gene | 3.0 | 0.50 | 2.4 | 0.18 |
| HBB | hemoglobin, beta | 3.0 | 0.50 | 2.6 | 0.18 |
| DUT | dUTP pyrophosphatase | 2.2 | 0.50 | 2.2 | 0.18 |
| RARRES1 | retinoic acid receptor responder 1 | −3.0 | 0.90 | −2.2 | 0.52 |
| Signal transduction | | | | | |
| PIK3R1 | phosphoinositide-3-kinase, reg subunit, | 3.1 | 0.50 | 2.3 | 0.18 |
| | polypeptide 1 | | | | |
| NPR3 | atrionatriuretic peptide receptor C | 3.1 | 0.50 | 2.5 | 0.18 |
| CBLB | Cas-Br-M ectropic retroviral transforming | 2.3 | 0.50 | 2.3 | 0.18 |
| | sequence b | | | | |
| EDNRA | endothelin receptor type A | 2.1 | 2.76 | 2.1 | 0.52 |
| DKFZp564I1922 | adlican | 2.0 | 1.28 | 2.4 | 0.18 |
| TNFRSF11B | tumor necrosis factor receptor superfamily, | −2.7 | 1.69 | −2.0 | 1.18 |
| | member 11b | | | | |
| SCYA2 | small inducible cytokine A2 | −3.5 | 0.90 | −2.9 | 0.18 |
| Metabolism | | | | | |
| EIF1AY | eukaryotic translation initiation factor 1A | 2.2 | 0.50 | 2.2 | 0.60 |
| KIAA0669 | KIAA0669 gene product | 2.2 | 0.50 | 3.2 | 0.18 |
| SFPQ | splicing factor proline/glutamine rich | 2.1 | 0.50 | 2.0 | 0.18 |
| Nucleus | | | | | |
| PHLDA1 | pleckstrin homology-like domain, family A, | 3.5 | 0.50 | 5.1 | 0.18 |
| | member 1 | | | | |
| PHLDA1 | pleckstrin homology-like domain, family A, | 3.3 | 0.50 | 4.9 | 0.18 |
| | member 1 | | | | |
| ANP32E | acidic nuclear phosphoprotein 32 family, | 2.0 | 0.50 | 2.7 | 0.18 |
| | member E | | | | |
| Cell adhesion/cell | | | | | |
| communication | | | | | |
| COL21A1 | collagen, type XXI, alpha 1 | 2.3 | 0.50 | 2.3 | 0.18 |
| FCN3 | ficolin 3 | −3.2 | 0.90 | −2.6 | 0.18 |
| Catalytic activity | | | | | |
| DBY | DEAD/H (Asp-Glu-Ala-Asp/His) box | 2.4 | 0.50 | 2.7 | 0.52 |
| | polypeptide | | | | |
| AGXT2L1 | alanine-glyoxylate aminotransferase 2-like 1 | −2.5 | 0.90 | −2.4 | 0.18 |
| Binding | | | | | |
| PEPP2 | phosphoinositol 3-phosphate-binding protein-2 | 2.2 | 0.50 | 2.4 | 0.18 |
| QKI | homolog of mouse quaking QKI | 2.1 | 0.50 | 2.0 | 0.18 |
| Other | | | | | |
| MYT1 | myelin transcription factor 1 | 2.0 | 0.90 | 2.4 | 0.18 |
| ASPN | asporin (LRR class 1) | 2.1 | 0.50 | 3.3 | 0.18 |
| MYH6 | myosin, heavy polypeptide 6, cardiac muscle, | −2.5 | 0.50 | −3.7 | 0.18 |
| | alpha | | | | |
| AF000381 | folate binding protein mRNA, partial cds. | 3.7 | 0.50 | 3.0 | 0.18 |
| TPR | translocated promoter region | 2.5 | 0.50 | 2.2 | 0.18 |
| none | Homo sapiens, clone IMAGE: 4182947, | 2.3 | 0.50 | 3.0 | 0.18 |
| | mRNA | | | | |
| none | Homo sapiens, clone IMAGE: 4182947, | 2.3 | 0.50 | 3.1 | 0.18 |
| | mRNA | | | | |
| none | Homo sapiens, clone IMAGE: 3611719, | 2.2 | 0.50 | 2.1 | 0.18 |
| | mRNA | | | | |
| none | Homo sapiens cDNA FLJ11918 fis | 2.2 | 0.50 | 2.8 | 0.18 |
| P311 | similar to Neuronal protein 3.1 | 2.1 | 0.90 | 2.4 | 0.18 |
| none | Human clone 23589 mRNA sequence | 2.1 | 0.90 | 2.6 | 0.18 |
| HMG2 | high-mobility group (nonhistone | 2.1 | 0.50 | 3.1 | 0.18 |
| | chromosomal) protein 2 | | | | |
| SERPINA3 | serine (or cysteine) proteinase inhibitor, clade | −2.5 | 0.50 | −2.0 | 0.18 |
| | A, mem 3 | | | | |

*Fold change described the mean gene expression for ischemic and nonischemic samples relative to nonfailing samples. FDR is false discovery rate, analogous to a p value (as a percentage) adjusted for multiple comparisons. NICM-NF denotes comparison between nonfailing hearts and nonischemic cardiomyopathy samples ICM-NF denotes comparison between nonfailing hearts and ischemic cardiomyopathy samples

TABLE 3
Differentially expressed genes(n = 216) unique to the nonischemic-
cardiomyopathy versus-nonfailing-heart comparison*

| | | | |
|---|---|---|---|
| | | Fold | |
| Gene symbol | Gene Name | change* | FDR |

Metabolism

| | | | |
|---|---|---|---|
| FACL3 | Acyl-CoA synthetase long-chain family member 3 | 2.8 | 0.18 |
| HNRPH3 | Heterogeneous nuclear ribonucleoprotein H3 | 2.7 | 0.18 |
| FLJ22222 | Hypothetical protein FLJ22222 (protein | 2.6 | 0.18 |
| | metabolism | | |
| OSBPL8 | oxysterol binding protein-like 8 | 2.6 | 0.18 |
| ACE2 | angiotensin 1 converting enzyme 2 | 2.6 | 0.18 |
| VDU1 | pPVHL-interacting deubiquitinating enzyme 1 | 2.4 | 0.18 |
| LIPA | lipase A, lysosomal acid, cholesterol esterase | 2.4 | 0.18 |
| MGEA5 | meningioma expressed antigen 5 | 2.4 | 0.18 |
| FLJ12552 | hypothetical protein FLJ12552 | 2.4 | 0.18 |
| CPE | carboxypeptidase E | 2.4 | 0.18 |
| PLOD2 | procollagen-lysine, 2-oxoglutarate 5-dioxygenase 2 | 2.4 | 0.18 |
| SFRS7 | splicing factor, arginine/serine-rich 7 | 2.3 | 0.18 |
| YT521 | splicing factor YT521-B, K1AA1966 | 2.3 | 0.18 |
| SMARCA2 | SWI/SNF related, matrix assoc, actin dep reg of | 2.3 | 0.18 |
| | chromatin | | |
| GLS | glutaminase | 2.3 | 0.18 |
| CTSB | cathepsin B | 2.3 | 0.18 |
| RNASE4 | ribonuclease, Rnase A family, 4 | 2.3 | 0.18 |
| DPYD | dihyrophyrimidine dehydrogenase | 2.3 | 0.18 |
| GATM | glycine amidinotransferase | 2.3 | 0.18 |
| HSP105B | heat shock 105 kD | 2.2 | 0.18 |
| GATM | glycine amidinotransferase | 2.2 | 0.18 |
| PIGK | phosphatidylinositol glycan, class K | 2.2 | 0.18 |
| DNAJB4 | DnaJ (Hsp40) homolog, subfamily B, member 4 | 2.2 | 0.18 |
| BACE | beta-site APP-cleaving enzyme | 2.2 | 0.18 |
| NBS1 | nijmegen breakage syndrome 1 | 2.1 | 0.18 |
| LUC7A | cisplatin resistance-associated overexpressed | 2.1 | 0.18 |
| | protein | | |
| UBE1C | ubiquitin-activating enzyme E1C | 2.1 | 0.18 |
| GCH1 | GTP cyclohydrolase 1 | 2.0 | 1.2 |
| C15orf15 | chromosome 15 open reading frame 15 | 2.0 | 0.18 |
| FBXO3 | F-box only protein 3 | 2.0 | 0.18 |
| ODC1 | ornithine decarboxylase 1 | 2.0 | 0.18 |
| B3GALT3 | UDP-Gal:betaGcNAc beta 1,3- | 2.0 | 0.52 |
| | galactosyltransferase, polypeptide 3 | | |
| SEPP1 | selenoprotein P, plasma, 1 | 2.0 | 0.18 |
| SLC39A8 | solute carrier family 39, member 8 | −2.0 | 0.18 |

Cell growth/maintenance

| | | | |
|---|---|---|---|
| NAP1L3 | nucleosome assembly protein 1-like 3 | 3.1 | 0.18 |
| ARID4B | AT rich interactive domain 4B | 2.5 | 0.18 |
| CDKN1B | cyclin-dependant kinase inhibitor 1B | 2.5 | 0.18 |
| RNPC2 | RNA-binding region containing 2 | 2.4 | 0.18 |
| DUSP6 | dual specificity phosphatase 6 | 2.3 | 0.18 |
| NAP1L1 | nucleosome assembly protein 1-like 1 | 2.3 | 0.18 |
| DENR | density-regulated protein | 2.3 | 0.18 |
| CENTB2 | centaurin, beta 2 | 2.2 | 0.18 |
| TOB1 | transducer of ERBB2, 1 | 2.2 | 0.18 |
| SEC23A | Sec23 homolog A | 2.2 | 0.18 |
| SNAP23 | synaptosomal-associated protein, 23 kD | 2.2 | 0.18 |
| ID4 | inhibitor of DNA binding 4, dominant negative | 2.2 | 0.18 |
| | helix-loop-helix protein | | |
| SEC24B | SEC24 related gene family, member B | 2.2 | 0.18 |
| CGI-142 | hepatoma-derived growth factor 2 | 2.2 | 0.18 |
| BM11 | B lymphoma Mo-MLV insertion region | 2.2 | 0.18 |
| ABCA8 | ATP-binding cassette, sub-family A, member 8 | 2.1 | 0.18 |
| BC003689 | high-mobility group nucleosomal binding domain 2 | 2.1 | 0.18 |
| GAPCENA | rab6 GTPase activating protein | 2.1 | 0.18 |
| PURA | purine-rich element binding protein A | 2.1 | 0.18 |
| NUP153 | Nucleoporin 153 kD | 2.1 | 0.18 |
| PLSCR4 | phospholipids scramblase 4 | 2.1 | 0.18 |
| NAB1 | NGFI-A binding protein 1 | 2.1 | 0.18 |
| TRIM33 | tripartite motif-containing 33 | 2.1 | 0.18 |
| DSIPI | delta sleep inducing peptide, immunoreactor | 2.1 | 0.18 |
| CTBP2 | C-terminal binding protein 2 | 2.1 | 0.18 |
| JJAZ1 | Joined to JAZF1 | 2.1 | 0.18 |
| ZFHX1B | zinc finger homeobox 1b | 2.0 | 0.18 |
| ZNF161 | zinc finger protein 161 | 2.0 | 0.18 |
| SERP1 | stress-associated endoplasmic reticulum protein 1 | 2.0 | 0.18 |

Signal transduction

| | | | |
|---|---|---|---|
| APM1 | adipocyte, C1Q and collagen domain containing | 3.5 | 0.52 |
| SH3BGRL | SH3-domain binding glutamic acid-rich protein like | 3.1 | 0.18 |
| ARH1 | ras homolog gene family, member I | 2.7 | 0.18 |
| ERBB2IP | erbb2 interacting protein | 2.6 | 0.18 |
| P23 | unactive progesterone receptor, 23 kD | 2.5 | 0.68 |
| SH3BP5 | SH3-domain binding protein 5 | 2.4 | 0.18 |
| GHR | growth hormone receptor | 2.4 | 0.18 |
| APP | amloid beta precursor protein | 2.4 | 0.18 |
| STAT1 | signal transducer an activator of transcription 1, | 2.4 | 0.18 |
| | 91 kD | | |
| TCF7L2 | transcription factor 7-like 2 | 2.4 | 0.18 |
| PDE4B | phosphodiesterase 4B, cAMP-specific | 2.3 | 0.52 |
| STC1 | stanniocalcin 1 | 2.3 | 0.52 |
| TGFBR3 | transforming growth factor, beta receptor III | 2.3 | 0.18 |
| LEPR | leptin receptor | 2.3 | 0.18 |
| PIK3CA | phosphoinositide-3-kinase, catalytic, alpha | 2.2 | 0.18 |
| | polypeptide | | |
| PENK | proenkephalin | 2.1 | 0.18 |
| ATP6IP2 | ATPase, H+ transporting, lysosomal interactin | 2.1 | 0.18 |
| | protein 2 | | |
| IGFBP3 | insulin-like growth factor binding protein 3 | 2.1 | 0.18 |
| ROCK1 | Rho-associated, coiled-coil containing protein | 2.1 | 0.18 |
| | kinase 1 | | |
| OGN | osteoglycin | 2.1 | 0.18 |
| LIM | LIM protein | 2.1 | 0.18 |
| AKAP11 | A kinase anchor protein 11 | 2.1 | 0.18 |
| TCF7L2 | transcription factor 7-like 2 | 2.1 | 0.18 |
| PDGFC | platelet derived growth factor C | 2.1 | 0.18 |
| NCOA2 | nuclear receptor coactivator 2 | 2.0 | 0.18 |

Binding

| | | | |
|---|---|---|---|
| K1AA0882 | K1AA0882 protein | 2.3 | 0.18 |
| TRIM22 | tripartite motif-containing 22 | 2.3 | 0.18 |
| K1AA0993 | WD repeat and FYVE domain containing 3 | 2.2 | 0.18 |
| BC017580 | stress 70 protein chaperone, microsome-associated, | 2.2 | 0.18 |
| | 60 kDa | | |
| SE70-2 | cutaneous T-cell lymphoma tumor antigen se70-2 | 2.2 | 0.18 |
| EPS15 | epidermal growth factor receptor pathway substrate | 2.2 | 0.18 |
| | 15 | | |
| MYCBP2 | MYC bindng protein 2, KIAA0916 | 2.2 | 0.18 |
| MATR3 | matrin 3 | 2.2 | 0.18 |
| PLAGL1 | pleiomorphic adenoma gene-like 1 | 2.1 | 0.18 |
| KIAA0853 | KIAA0853 protein | 2.1 | 0.18 |
| ZZZ3 | zinc finger, ZZ domain containing 3, | 2.1 | 0.18 |
| | DKFZP564I052 | | |
| MATR3 | matrin 3 | 2.1 | 0.18 |
| CRI1 | CREBBP/EP300 inhibitory protein 1 | 2.1 | 0.18 |
| FMR1 | fragile X mental retardation 1 | 3.3 | 0.18 |
| YY1 | YY1 transcription factor | 2.4 | 0.18 |
| SP3 | Sp3 transcription factor | 2.4 | 0.18 |
| RBBP1 | retinoblastoma binding protein 1 | 2.3 | 1.2 |
| NR2F2 | nuclear receptor subfamily 2, group F, member 2 | 2.3 | 0.18 |
| SOX4 | SRY (sex determining region Y)-box 4 | 2.3 | 0.18 |
| STAT4 | signal transducer and activator of transcription 4 | 2.1 | 0.18 |
| ELK3 | ELK3, ETS-domain protein | 2.1 | 0.18 |

Inflammation/immune response

| | | | |
|---|---|---|---|
| HF1 | H factor 1 (complement) | 2.5 | 0.18 |
| NR3C1 | nuclear receptor subfamily 3, group C, member 1 | 2.1 | 0.18 |
| HLA-DPA1 | major histocompatibility complex, class II, DP | 2.3 | 0.18 |
| | alpha 1 | | |
| IL27 | interleukin 27 | 2.0 | 0.52 |

Development

| | | | |
|---|---|---|---|
| LUM | lumican | 2.8 | 0.18 |
| FRZB | frizzled-related protein | 2.1 | 0.18 |
| DIXDC1 | DIX domain containing 1, K1AA1735 | 2.0 | 0.18 |
| ATP2C1 | ATPase, Ca++ transporting, type 2C, member 1 | 2.0 | 0.18 |
| OSF-2 | periostin, osteoblast specific factor | 3.0 | 0.18 |

Cell adhesion

| | | | |
|---|---|---|---|
| PNN | pinin, desmosome associated protein | 2.3 | 0.68 |
| LAMB1 | laminin, beta 1 | 2.3 | 0.18 |
| DPT | dermatopontin | 2.2 | 0.18 |

Catalytic activity

| | | | |
|---|---|---|---|
| HNMT | histamine N-methyltransferase | 2.2 | 0.18 |
| HS2ST1 | heparin sulfate 2-O-sulfotransferase 1 | 2.1 | 0.18 |
| PHKB | phosphorylase kinase, beta | 2.1 | 0.18 |
| DKFZP586A0522 | DKFZP586A0522 protein | 2.0 | 0.18 |

Apostosis

| | | | |
|---|---|---|---|
| BNIP3L | BCL2/adenovirus E1B 19 kD interacting protein 3- | 2.2 | 0.18 |
| | like | | |
| SPF30 | survival motor neuron domain containing 1 | 2.2 | 0.18 |
| TIA1 | TIA1 cytotoxic granule-associated RNA binding | 2.1 | 0.18 |
| | protein | | |
| BCL2 | B-cell CLL/lymphoma 2 | 2.0 | 0.18 |

Cytoskeleton

| | | | |
|---|---|---|---|
| DMD | dystrophin | 2.2 | 0.18 |
| ADD3 | adducing 3 | 2.1 | 0.18 |
| KLHL2 | kelch-like 2, Mayven | 2.0 | 0.18 |

Other

| | | | |
|---|---|---|---|
| KTN1 | kinectin 1 (kinesin receptor) | 2.7 | 0.18 |
| C8orf2 | chromosome 8 open reading frame 2 | 2.2 | 0.18 |
| GCC2 | GRIP and coiled-coil domain containing 2, | 2.0 | 0.18 |
| | KIAA0336 | | |
| AF054589 | I-mfa domain-containing protein | 2.2 | 0.18 |
| EFA6R | ADP-ribosylation factor guanine nucleotide factor 6 | 3.0 | 0.18 |
| AF130089 | Homo sapiens clone FLB9440 PRO2550 mRNA, | 2.9 | 0.18 |
| | complete cds. | | |
| AF130082 | Homo sapiens clone FLC1492 PRO3121 mRNA, | 2.9 | 0.18 |
| | complete cds. | | |
| AF070641 | Homo sapiens clone 24421 mRNA sequence | 2.7 | 0.18 |
| AF271775 | Homo sapiens DC49 mRNA, complete cds. | 2.7 | 0.18 |
| CG005 | phosphonoformate immuno-associated protein 5 | 2.6 | 0.18 |
| KIDINS220 | likely homolog of rat kinase D-interacting | 2.6 | 0.18 |
| | substance of 220 kDa | | |
| ALEX3 | ALEX3 protein | 2.5 | 0.18 |
| KIAA0680 | chromosome 6 open reading frame 56 | 2.5 | 0.18 |
| FLJ11273 | hypothetical protein FLJ11273 | 2.4 | 0.18 |
| UBQLN2 | ubiquilin 2 | 2.4 | 0.18 |
| DICER1 | Dicer1, Dcr-1 homolog (Drosophila) | 2.4 | 0.18 |
| RYBP | RING1 and YY1 binding protein | 2.4 | 0.18 |
| TEB4 | similar to S. cerevisiae SSM4 | 2.3 | 0.18 |
| IPW | imprinted in Prader-Willi syndrome | 2.3 | 0.52 |
| PRKAR2B | protein kinase, cAMP-dependent, regulatory, type | 2.3 | 0.18 |
| | II, beta | | |
| SP329 | likely ortholog of mouse modulator of KLF7 | .3 | 0.18 |
| | activity | | |
| SDCCAG1 | serologically defined colon cancer antigen 1 | 2.3 | 0.52 |
| MARCKS | myristoylated alanine-rich protein kinase C | 2.3 | 0.18 |
| | substrate | | |
| AK027252 | Homo sapiens clone 23664 and 23905 mRNA | 2.3 | 0.18 |
| | sequence | | |
| EPS8 | epidermal growth factor receptor pathway substrate 8 | 2.3 | 0.18 |
| AK055910 | Homo sapiens cDNA FLJ31348 fis, clone | 2.2 | 0.18 |
| | MESAN2000026 | | |
| KIAA0143 | KIAA0143 protein | 2.2 | 0.18 |
| AK025583 | Homo sapiens cDNA clone | 2.2 | 0.18 |
| KIAA0914 | family with sequence similarity 13, member A1 | 2.2 | 0.18 |
| STAG2 | stromal antigen 2 | 2.2 | 0.18 |
| AL136139 | Contains 3′ part of the gene for enhancer of | 2.2 | 0.18 |
| | filamentation (HEF1) | | |
| M55536 | Human glucose transporter pseudogene | 2.2 | 0.18 |
| AASDHPPT | aminoadipate-semialdehyde dehydrogenase- | 2.2 | 0.18 |
| | phosphopantetheinyl | | |
| PTN | pleiotrophin | 2.2 | 0.18 |
| MGC4276 | HESB like domain containing 2 | 2.2 | 0.18 |
| LOC51110 | lactamase, beta 2 | 2.2 | 0.18 |
| GATA6 | GATA binding protein 6 | 2.2 | 0.18 |
| AK021980 | Homo sapiens cDNA FLJ11918 fis, clone | 2.2 | 0.18 |
| | HEMBB1000272 | | |
| AK025216 | Homo sapiens cDNA: FLJ21563 fis, clone | 2.2 | 0.18 |
| | COL06445 | | |
| none | chromosome 6 open reading frame 111: | 2.2 | 0.18 |
| | DKFZp564B0769 | | |
| AK021980 | Homo sapiens cDNA FLJ11918 fis, clone | 2.2 | 0.18 |
| | HEMBB1000272 | | |
| 13CDNA73 | hypothetical protein CG003 | 2.1 | 0.18 |
| GASP | G protein-coupled receptor-associated sorting | 2.1 | 0.18 |
| | protein, K1Aaa0443 | | |
| PSIP2 | PC4 and SFRS1 interacting protein 2 | 2.1 | 0.18 |
| ARL5 | ADP-ribosylation factor-like 5 | 2.1 | 0.18 |
| K1AA0582 | K1AA0582 protein | 2.1 | 0.18 |
| FLJ23018 | hypothetical protein FLJ23018 | 2.1 | 0.18 |
| none | hypothetical protein DKFZp761K1423 | 2.1 | 0.52 |
| STAG2 | stromal antigen 2 | 2.1 | 0.18 |
| SACS | spastic ataxia of Charlevoix-Saguenay (sacsin) | 2.1 | 0.18 |
| AW190289 | ESTs | 2.1 | 0.18 |
| KIAA1109 | KIAA1109 protein | 2.1 | 0.18 |
| KPNB3 | karyopherin (importin) beta 3 | 2.1 | 0.18 |
| TTC3 | tetratricopeptide repeat domain | 2.1 | 0.18 |
| AK055600 | Homo sapiens mRNA; cDNA DKFZp434G012 | 2.1 | 0.18 |
| HHL | expressed in hematopoietic cells, heart, liver, | 2.1 | 0.18 |
| | KIAA0471 | | |
| RCP | Rab coupling protein | 2.1 | 0.18 |
| FLJ22104 | hypothetical protein FLJ22104 | 2.1 | 0.18 |
| BTN3A3 | butyrophilin, subfamily 3, member A3 | 2.1 | 0.18 |
| BCMP1 | transmembrane 4 superfamily member 10 | 2.1 | 0.18 |
| AV712064 | EST: Homo sapiens cDNA: DCAAUD05, 5′end, | 2.1 | 0.18 |
| | human dendrites | | |
| RNF38 | ring finger protein 38, FLJ21343 | 2.1 | 0.18 |
| AL049998 | Homo sapiens mRNA; cDNA DKEZp564L222 | 2.1 | 0.18 |
| HS696H22 | Human DNA sequence from clone RP4-696H22 | 2.1 | 0.18 |
| BC007568 | Homo sapiens, clone IMAGE: 3028427, mRNA, | 2.1 | 0.18 |
| | partial cds | | |
| DICER1 | Dicer 1, DCR-1 homolog (Drosophila) | 2.1 | 0.18 |
| HS21C048 | Homo sapiens chromosome 21 segment HS21C048 | 2.1 | 0.18 |
| XPO1 | exportin 1 (CRM1 homolog, yeast) | 2.0 | 0.18 |
| ALEX1 | ALEX1 protein | 2.0 | 0.18 |
| KIAA0372 | KIAA0372 gene product | 2.0 | 0.18 |
| DC8 | DKFZP566O1646 protein | 2.0 | 0.60 |
| FAM3C | family with sequence similarity 3, member C, | 2.0 | 0.18 |
| | GS3786 | | |
| AL713745 | Homo sapiens mRNA; cDNA DKFZp761J0523 | 2.0 | 0.18 |
| TTC3 | tetratricopeptide repeat domain 3 | 2.0 | 0.18 |
| TTC3 | tetratricopeptide repeat domain 3 | 2.0 | 0.18 |
| UNC84A | unc-84 homolog A (C. elegans), K1AA0810 | 2.0 | 0.18 |
| OAZIN | ornithine decarboxylase antizyme inhibitor | 2.0 | 0.18 |
| ZNF292 | ZNF292 zinc finger protein 292, K1AA0530 | 2.0 | 0.18 |
| PJA2 | praja 2, RING-H2 motif containing, K1AA0438 | 2.0 | 0.18 |
| HNRPA3 | heterogeneous nuclear ribonucleoprotein A3 | 2.0 | 0.18 |
| HS73M23 | ESTs | 2.0 | 0.18 |
| RECQL | RecQ protein-like (DNA helicase Q1-like) | 2.0 | 0.18 |
| DR1 | down-regular of transcription 1, TBP-binding | 2.2 | 0.18 |
| | (negative cofactor 2) | | |
| AL049437 | Homo sapiens mRNA; cDNA DKFZp586E1120 | 2.2 | 0.18 |

*Fold change described the mean gene expression for ischemic and nonischemic samples relative to nonfailing samples.
FDR is false discovery rate, analogous to a p value (as a percentage) adjusted for multiple comparisons.

TABLE 4
Diffeentially expressed genes (n = 31) unique to the
ischemic-cardiomyopathy-versus-nonfailing heart comparison*

| | | | |
|---|---|---|---|
| | | Fold | |
| Gene Symbol | Gene Name | change* | FDR |

cell growth/maintenance

| | | | |
|---|---|---|---|
| RPS4Y | ribosomal protein S4, Y-linked | 2.4 | 0.50 |
| ALS2CR3 | amyotrophic lateral sclerosis 2 chromosome | 2.3 | 0.50 |
| | region, candidate 3 | | |
| KPNB2 | karyopherin beta 2 | 2.1 | 0.50 |
| SLC16A7 | solute carrier family 16, member 17 | 2.1 | 0.50 |
| ZNF145 | zinc finger protein 145 | 2.1 | 1.1 |

Catalytic activity

| | | | |
|---|---|---|---|
| SERPINB1† | serine (or cysteine) proteinase inhibitor, clade B, | −2.2 | 0.90 |
| | member 1 | | |
| SERPINB1† | serine (or cysteine) proteinase inhibitor, clade B, | −2.2 | 2.4 |
| | member 1 | | |
| ATP1B3 | ATPase, Na+/K+ transporting, beta 3 polypeptide | −2.3 | 0.90 |
| SERPIINE1 | serine (or cysteine) proteinase inhibitor, clade E, | −2.3 | 3.0 |
| | member 1 | | |

signal transduction

| | | | |
|---|---|---|---|
| NPPB | natriuretic peptide precursor B | 4.4 | 2.8 |
| HSCDDANF | Human cardiodilatin-atrial natriuretic factor | 2.3 | 3.8 |
| PBEF | pre-B-cell colony-enhancing factor | −2.1 | 3.7 |

Transcription factors

| | | | |
|---|---|---|---|
| ATF3 | activating transcription factor 3 | −2.6 | 3.0 |
| SMAP31 | homeodomain-only protein | −3.3 | 0.90 |
| PTX3 | pentaxin-related gene, rapidly induced by IL-1 | −2.2 | 3.0 |
| | beta | | |
| S100A8 | S100 calcium binding protein A8 (calgranulin A) | −2.7 | 0.90 |

Development

| | | | |
|---|---|---|---|
| AR1H2 | ariadne homolog 2 | 2.0 | 0.50 |
| DLK1 | delta-like 1 homolog | 2.0 | 2.9 |

Metabolism

| | | | |
|---|---|---|---|
| PLA2G2A | phospholipase A2, group IIA | −3.4 | 0.90 |

Cytoskeleton

| | | | |
|---|---|---|---|
| MYL4 | myosin, light polypeptide 4, alkali; atrial, | 2.4 | 2.0 |
| | embryonic | | |

Other

| | | | |
|---|---|---|---|
| AF116676 | EMBL: Homo sapiens PRO1957 mRNA, | 2.3 | 2.4 |
| | complete cds. | | |
| TXNIP | thioredoxin ineracting protein | 2.3 | 0.50 |
| SYNPO2L | synaptopodin 2-like | 2.1 | 0.50 |
| FLJ11539 | hypothetical protein FLJ11539 | 2.1 | 0.50 |
| FLJ10159 | hypothetical protein FLJ10159 | 2.0 | 0.50 |
| none | Homo sapiens cDNA FLJ11918 fis, clone | 2.0 | 0.50 |
| | HEMBB1000272 | | |
| DKFZP434F0318 | hypothetical protein DKFZp434F0318 | 2.0 | 2.0 |
| none | Homo sapiens cDNA: FLJ22179 fis, clone | 2.0 | 0.50 |
| | HRC00920 | | |
| CD163 | CD163 antigen | −2.0 | 0.90 |
| none | Homo sapiens cDNA FLJ30298 fis, clone | −2.0 | 0.90 |
| | BRACE2003172 | | |
| none | Homo sapiens cDNA: FLJ21545 fis, clone | −3.0 | 0.90 |
| | COL06195 | | |

*Fold change described the mean gene expression for ischemic and nonischemic samples relative to nonfailing samples.
FDR is false discovery rate, analogous to a p value (as a percentage) adjusted for multiple comparisons.
†There are two entries for this gene product because it was identified as differentially expressed with two unique Affymetrix accession numbers.

TABLE 5
Differentially expressed genes common to previously published reports
Fold Change

| | Our Study | Our Study | | | | |
|---|---|---|---|---|---|---|
| Gene symbol | NICM-NF | ICM-NF | Tan(8) | Barrans (1) | Yung (9) | Steenman (7) |
| PHLDA1 | 5.1 | 3.5 | | | 5.43 | |
| PIK3R1 | 2.3 | 3.1 | | | | 2.73 |
| TPR | 2.2 | 2.5 | | | 2.02 | |
| COL21A1 | 2.3 | 2.3 | | | 3.52 | |
| EIF1AY | 2.2 | 2.2 | | 1.78 | | |
| MYH6 | −3.7 | −2.5 | | | −5.3 | −1.36 |
| FCN3 | −2.6 | −3.2 | | | −7.7 | |
| NPPB | | 4.4 | 3.3 | | | 7.24 |
| MYL4 | | 2.4 | | 2.01 | | 3.79 |
| HSCDDANF | | 2.3 | 4.2 | 19.5 | | 4.83 |
| ZNF145 | | 2.1 | | | | 2.33 |
| ATP1B3 | | −2.3 | | | −2.7 | |
| PLA2G2A | | −3.4 | −5.1 | | | |
| FMR1 | 3.3 | | | | 2.06 | |
| SH3BGRL | 3.1 | | | | | 1.20 |
| OSF-2 | 3.0 | | 12 | 1.96 | | |
| LUM | 2.8 | | 3.8 | | | |
| HNRPH3 | 2.7 | | | | 1.83 | |
| HF1 | 2.5 | | | 1.23 | | |
| CDKN1B | 2.5 | | | | 2.03 | |
| PDE4B | 2.3 | | | 2.41 | | |
| PTN | 2.2 | | | | 3.29 | |
| ATP6IP2 | 2.1 | | | 1.19 | | |
| GAPCENA | 2.1 | | | | 1.74 | |
| TIA1 | 2.1 | | | 2.14 | | |
| PLAGL1 | 2.1 | | | | 2.2 | |
| NR3C1 | 2.1 | | | | 1.72 | |
| DSIP1 | 2.1 | | | | | 1.29 |
| FBX03 | 2.0 | | | | 1.59 | |
| ODC1 | 2.0 | | | | 2.52 | |

Gene symbols correspond to gene products as noted in Tables 3-5.

TABLE 6

| Probe Set | Gene | |
|---|---|---|
| ID | Symbol | Gene Title |
| 202133_at | WWTR1 | WW domain containing transcription |
| | | regulator 1 |
| 202237_at | NNMT | nicotinamide N-methyltransferase |
| 211074_at | FOLR1 | folate receptor 1 (adult) /// folate |
| | | receptor 1 (adult) |
| 212190_at | SERPINE2 | serpin peptidase inhibitor, clade E (nexin, |
| | | plasminogen activator inhibitor type 1), |
| | | member 2 |
| 213102_at | ACTR3 | ARP3 actin-related protein 3 homolog |
| | | (yeast) |
| 213168_at | SP3 | Sp3 transcription factor |
| 215427_s_at | ZCCHC14 | zinc finger, CCHC domain containing 14 |

### Reference List

1. 2004 Bioconductor Homepage [Online]. 2004.
2. Barrans J D, Allen P D, Stamatiou D, Dzau V J and Liew C C. Global gene expression profiling of end-stage dilated cardiomyopathy using a human cardiovascular-based cDNA microarray. Am J Pathol 160: 2035-2043, 2002.
3. Blaxall B C, Tschannen-Moran B M, Milano C A and Koch W J. Differential gene expression and genomic patient stratification following left ventricular assist device support. J Am Coll Cardiol 41: 1096-1106, 2003.
4. Boehm M and Nabel E G. Angiotensin-converting enzyme 2—a new cardiac regulator. N Engl J Med 347: 1795-1797, 2002.
5. Boheler K R, Volkova M, Morrell C, Garg R, Zhu Y, Margulies K, Seymour A M and Lakatta E G. Sex- and age-dependent human transcriptome variability: implications for chronic heart failure. Proc Natl Acad Sci USA 100: 2754-2759, 2003.
6. Bullinger L, Dohner K, Bair E, Frohling S, Schlenk R F, Tibshirani R, Dohner H and Pollack J R. Use of gene-expression profiling to identify prognostic subclasses in adult acute myeloid leukemia. N Engl J Med 350: 1605-1616, 2004.
7. Butte A. The use and analysis of microarray data. Nat Rev Drug Discov 1: 951-960, 2002.
8. Califf R M, Adams K F, McKenna W J, Gheorghiade M, Uretsky B F, McNulty S E, Darius H, Schulman K, Zannad F, Handberg-Thurmond E, Harrell F E, Jr., Wheeler W, Soler-Soler J and Swedberg K. A randomized controlled trial of epoprostenol therapy for severe congestive heart failure: The Flolan International Randomized Survival Trial (FIRST). Am Heart J 134: 44-54, 1997.
9. Chaitman B R, Pepine C J, Parker J O, Skopal J, Chumakova G, Kuch J, Wang W, Skettino S L and Wolff A A. Effects of ranolazine with atenolol, amlodipine, or diltiazem on exercise tolerance and angina frequency in patients with severe chronic angina: a randomized controlled trial. JAMA 291: 309-316, 2004.
10. Chen M M, Ashley E A, Deng D X, Tsalenko A, Deng A, Tabibiazar R, Ben Dor A, Fenster B, Yang E, King J Y, Fowler M, Robbins R, Johnson F L, Bruhn L, McDonagh T, Dargie H, Yakhini Z, Tsao P S and Quertermous T. Novel role for the potent endogenous inotrope apelin in human cardiac dysfunction. Circulation 108: 1432-1439, 2003.
11. Chen Y, Park S, Li Y, Missov E, Hou M, Han X, Hall J L, Miller L W and Bache R J. Alterations of gene expression in failing myocardium following left ventricular assist device support. Physiol Genomics 14: 251-260, 2003.
12. Chung E S, Packer M, Lo K H, Fasanmade A A and Willerson J T. Randomized, double-blind, placebo-controlled, pilot trial of infliximab, a chimeric monoclonal antibody to tumor necrosis factor-alpha, in patients with moderate-to-severe heart failure: results of the anti-TNF Therapy Against Congestive Heart Failure (ATTACH) trial. Circulation 107: 3133-3140, 2003.
13. CIBIS-II Investigators and Committees. The Cardiac Insufficiency Bisoprolol Study II (CIBIS-II): a randomised trial. Lancet 353: 9-13, 1999.
14. Colucci W S and Braunwald E. Pathophysiology of Heart Failure. In: Heart Disease: A Textbook of Cardiovascular Medicine, edited by Braunwald E, Zipes D and Libby P. Philadelphia: W.B. Saunders, 2004.
15. Cook S A and Rosenzweig A. DNA microarrays: implications for cardiovascular medicine. Circ Res 91: 559-564, 2002.
16. Costanzo-Nordin M R, O'Connell J B, Engelmeier R S, Moran J F and Scanlon P J. Dilated cardiomyopathy: functional status, hemodynamics, arrhythmias, and prognosis. Cathet Cardiovasc Diagn 11: 445-453, 1985.
17. Crackower M A, Sarao R, Oudit G Y, Yagil C, Kozieradzki I, Scanga S E, Oliveira-dos-Santos A J, da Costa J, Zhang L, Pei Y, Scholey J, Ferrario C M, Manoukian A S, Chappell M C, Backx P H, Yagil Y and Penninger J M. Angiotensin-converting enzyme 2 is an essential regulator of heart function. Nature 417: 822-828, 2002.
18. Day M L, Schwartz D, Wiegand R C, Stockman P T, Brunnert S R, Tolunay H E, Currie M G, Standaert D G and Needleman P. Ventricular atriopeptin. Unmasking of messenger RNA and peptide synthesis by hypertrophy or dexamethasone. Hypertension 9: 485-491, 1987.
19. Doval H C, Nul D R, Grancelli H O, Perrone S V, Bortman G R and Curiel R. Randomised trial of low-dose amiodarone in severe congestive heart failure. Grupo de Estudio de la Sobrevida en la Insuficiencia Cardiaca en Argentina (GESICA). Lancet 344: 493-498, 1994.
20. Draghici S. Statistical intelligence: effective analysis of high-density microarray data. Drug Discovery Today 7: S55-S63, 2002.
21. Dries D L, Sweitzer N K, Drazner M H, Stevenson L W and Gersh B J. Prognostic impact of diabetes mellitus in patients with heart failure according to the etiology of left ventricular systolic dysfunction. J Am Coll Cardiol 38: 421-428, 2001.
22. Eisen M B, Spellman P T, Brown P O and Botstein D. Cluster analysis and display of genome-wide expression patterns. Proc Natl Acad Sci USA 95: 14863-14868, 1998.
23. Felker G M, Benza R L, Chandler A B, Leimberger J D, Cuffe M S, Califf R M, Gheorghiade M and O'Connor C M. Heart failure etiology and response to milrinone in decompensated heart failure: results from the OPTIME-CHF study. J Am Coll Cardiol 41: 997-1003, 2003.
24. Felker G M, Thompson R E, Hare J M, Hruban R H, Clemetson D E, Howard D L, Baughman K L and Kasper E K. Underlying causes and long-term survival in patients with initially unexplained cardiomyopathy. N Engl J Med 342: 1077-1084, 2000.
25. Hall J L, Grindle S, Han X, Fermin D, Park S, Chen Y, Bache R J, Mariash A, Guan Z, Ormaza S, Thompson J, Graziano J, Sam Lazaro S E, Pan S, Simari R D and Miller L W. Genomic Profiling of the Human Heart Before and After Mechanical Support with a Ventricular Assist Device Reveals Alterations in Vascular Signaling Networks. Physiol Genomics 2004.
26. Hare J M, Walford G D, Hruban R H, Hutchins G M, Deckers J W and Baughman K L. Ischemic cardiomyopathy: endomyocardial biopsy and ventriculographic evaluation of patients with congestive heart failure, dilated cardiomyopathy and coronary artery disease. J Am Coll Cardiol 20: 1318-1325, 1992.
27. Hunt S A, Baker D W, Chin M H, Cinquegrani M P, Feldmanmd A M, Francis G S, Ganiats T G, Goldstein S, Gregoratos G, Jessup M L, Noble R J, Packer M, Silver M A, Stevenson L W, Gibbons R J, Antman E M, Alpert J S, Faxon D P, Fuster V, Gregoratos G, Jacobs A K, Hiratzka L F, Russell R O and Smith S C, Jr. ACC/AHA Guidelines for the Evaluation and Management of Chronic Heart Failure in the Adult: Executive Summary A Report of the American College of Cardiology/American Heart Association Task Force on Practice Guidelines (Committee to Revise the 1995 Guidelines for the Evaluation and Management of Heart Failure): Developed in Collaboration With the International Society for Heart and Lung Transplantation; Endorsed by the Heart Failure Society of America. Circulation 104: 2996-3007, 2001.
28. Hwang J J, Allen P D, Tseng G C, Lam C W, Fananapazir L, Dzau V J and Liew C C. Microarray gene expression profiles in dilated and hypertrophic cardiomyopathic end-stage heart failure. Physiol Genomics 10: 31-44, 2002.
29. Irizarry R A, Bolstad B M, Collin F, Cope L M, Hobbs B and Speed T P. Summaries of Affymetrix GeneChip probe level data. Nucleic Acids Res 31: e15, 2003.
30. Kaab S, Barth A S, Margerie D, Dugas M, Gebauer M, Zwermann L, Merk S, Pfeufer A, Steinmeyer K, Bleich M, Kreuzer E, Steinbeck G and Nabauer M. Global gene expression in human myocardium-oligonucleotide microarray analysis of regional diversity and transcriptional regulation in heart failure. J Mol Med 82: 308-316, 2004.
31. Khan T A, Bianchi C, Voisine P, Feng J, Baker J, Hart M, Takahashi M, Stahl G and Sellke F W. Reduction of myocardial reperfusion injury by aprotinin after regional ischemia and cardioplegic arrest. Journal of Thoracic and Cardiovascular Surgery 128: 602-608, 2004.
32. Kittleson M, Hurwitz S, Shah M R, Nohria A, Lewis E, Givertz M, Fang J, Jarcho J, Mudge G and Stevenson L W. Development of circulatory-renal limitations to angiotensin-converting enzyme inhibitors identifies patients with severe heart failure and early mortality. J Am Coll Cardiol 41: 2029-2035, 2003.
33. Kittleson M M, Ye S Q, Irizarry R A, Minhas K M, Edness G, Conte J V, Parmigiani G, Miller L W, Chen Y, Hall J L, Garcia J G N and Hare J M. Identification of a Gene Expression Profile That Differentiates Between Ischemic and Nonischemic Cardiomyopathy. Circulation 110: 3444-3451, 2004.
34. Levine B, Kalman J, Mayer L, Fillit H M and Packer M. Elevated circulating levels of tumor necrosis factor in severe chronic heart failure. N Engl J Med 323: 236-241, 1990.
35. Liu E T and Karuturi K R. Microarrays and clinical investigations. N Engl J Med 350: 1595-1597, 2004.
36. Lowes B D, Minobe W, Abraham W T, Rizeq M N, Bohlmeyer T J, Quaife R A, Roden R L, Dutcher D L, Robertson A D, Voelkel N F, Badesch D B, Groves B M, Gilbert E M and Bristow M R. Changes in gene expression in the intact human heart. Downregulation of alpha-myosin heavy chain in hypertrophied, failing ventricular myocardium. J Clin Invest 100: 2315-2324, 1997.
37. Mann D L, McMurray J J, Packer M, Swedberg K, Borer J S, Colucci W S, Djian J, Drexler H, Feldman A, Kober L, Krum H, Liu P, Nieminen M, Tavazzi L, van Veldhuisen D J, Waldenstrom A, Warren M, Westheim A, Zannad F and Fleming T. Targeted anticytokine therapy in patients with chronic heart failure: results of the Randomized Etanercept Worldwide Evaluation (RENEWAL). Circulation 109: 1594-1602, 2004.
38. Peng X, Wood C L, Blalock E M, Chen K C, Landfield P W and Stromberg A J. Statistical implications of pooling RNA samples for microarray experiments. BMC Bioinformatics 4: 26, 2003.
39. Pokrovsky V M and Osadchiy O E. Regulatory peptides as modulators of vagal influence on cardiac rhythm. Can J Physiol Pharmacol 73: 1235-1245, 1995.
40. Rauchhaus M, Doehner W, Francis D P, Davos C, Kemp M, Liebenthal C, Niebauer J, Hooper J, Volk H D, Coats A J and Anker S D. Plasma cytokine parameters and mortality in patients with chronic heart failure. Circulation 102: 3060-3067, 2000.
41. Sarwal M, Chua M S, Kambham N, Hsieh S C, Satterwhite T, Masek M and Salvatierra O, Jr. Molecular heterogeneity in acute renal allograft rejection identified by DNA microarray profiling. N Engl J Med 349: 125-138, 2003.
42. Singh S N, Fletcher R D, Fisher S G, Singh B N, Lewis H D, Deedwania P C, Massie B M, Colling C and Lazzeri D. Amiodarone in patients with congestive heart failure and asymptomatic ventricular arrhythmia. Survival Trial of Antiarrhythmic Therapy in Congestive Heart Failure. N Engl J Med 333: 77-82, 1995.
43. Singhal S, Kyvemitis C G, Johnson S W, Kaiser L R, Liebman M N and Albelda S M. Microarray data simulator for improved selection of differentially expressed genes. Cancer Biol Ther 2: 383-391, 2003.
44. Steenman M, Chen Y W, Le Cunff M, Lamirault G, Varro A, Hoffman E and Leger J J. Transcriptomal analysis of failing and nonfailing human hearts. Physiol Genomics 12: 97-112, 2003.
45. Tan F L, Moravec C S, Li J, Apperson-Hansen C, McCarthy P M, Young J B and Bond M. The gene expression fingerprint of human heart failure. Proc Natl Acad Sci USA 99: 11387-11392, 2002.
46. Tibshirani R, Hastie T, Narasimhan B and Chu G. Diagnosis of multiple cancer types by shrunken centroids of gene expression. Proc Natl Acad Sci USA 99: 6567-6572, 2002.
47. Towbin J A and Bowles N E. Molecular genetics of left ventricular dysfunction. Curr Mol Med 1: 81-90, 2001.
48. Turner A J, Tipnis S R, Guy J L, Rice G and Hooper N M. ACEH/ACE2 is a novel mammalian metallocarboxypeptidase and a homologue of angiotensin-converting enzyme insensitive to ACE inhibitors. Can J Physiol Pharmacol 80: 346-353, 2002.
49. Tusher V G, Tibshirani R and Chu G. Significance analysis of microarrays applied to the ionizing radiation response. Proc Natl Acad Sci USA 98: 5116-5121, 2001.
50. Valk P J, Verhaak R G, Beijen M A, Erpelinck C A, Barjesteh van Waalwijk van Doom-Khosrovani, Boer J M, Beverloo H B, Moorhouse M J, van der Spek P J, Lowenberg B and Delwel R. Prognostically useful gene-expression profiles in acute myeloid leukemia. N Engl J Med 350: 1617-1628, 2004.
51. Yung C K, Halperin V L, Tomaselli G F and Winslow R L. Gene expression profiles in end-stage human idiopathic dilated cardiomyopathy: altered expression of apoptotic and cytoskeletal genes. Genomics 83: 281-297, 2004.
52. Barrans J D, Allen P D, Stamation D, Dzau V J and Liew C C. Global gene expression profiling of end-stage dilated cardiomyopathy using a human cardiovascular-based cDNA microarray. Am J Pathol 160: 2035-2043, 2002.
53. Chen Y, Park 5, Li Y, Missov E, lou M, Han X, Hall J L, Miller L W and Bache R J. Alterations of gene expression in failing myocardium following left ventricular assist device support. Physiol Genornics 14:25 1-260. 2003.
54. Felker G M, Shaw L K and O'Connor C M. A standardized definition of ischemic cardiomyopathy for use in clinical research. J Am Coil C'ardiol 39:2 10-218, 2002.
55. Hare J M, Walford G D, Hruban R H, Hutchins G M, Deckers J W and Baughman K L. Ischemic cardiomyopathy: endomyocardial biopsy and ventriculographic evaluation of patients with congestive heart failure, dilated cardiomyopathy and coronary artery disease. J Am Coil Cardiol 20: 13 18-1325, 1992.
56. Irizarry R A, Bolstad B M, Collin F, Cope L M, Hobbs B and Speed T P. Summaries of Affymetrix GeneChip probe level data. Nucleic Acids Res 31: el 5, 2003.
57. Irizarry R A, Hobbs B, Collin F, Beazer-Barclay Y D, Antoneilis K J, Scherf U and Speed T P. Exploration, normalization, and summaries of high density oligonucleotide array probe level data. Biostaiistics 4: 249-264, 2003.
58. Steenman M, Chen Y W, Le Cunff M, Lamirault C, Varro A, Hoffman E and Leger J. J. Transcriptomal analysis of failing and nonfailing human hearts. Physiol Genomics 12: 97-112, 2003.
59. Tan F L, Moravec C S, Li J, Apperson-Hansen C, McCarthy P M, Young J B and Bond M. The gene expression fingerprint of human heart failure. Proc Nail Acad Sci USA 99: 11387-11392, 2002.
60. Yung C K, Halperin V L, Tomaselli C F and Winslow R L. Gene expression profiles in end-stage human idiopathic dilated cardiomyopathy: altered expression of apoptotic and cytoskeletal genes. Genomics 83: 281-297, 2004.
61. Hunt S A, Abraham W T, Chin M H, Feldman A M, Francis G S, Ganiats T G et al. ACC/AHA 2005 Guideline Update for the Diagnosis and Management of Chronic Heart Failure in the Adult: a report of the American College of Cardiology/American Heart Association Task Force on Practice Guidelines (Writing Committee to Update the 2001 Guidelines for the Evaluation and Management of Heart Failure): developed in collaboration with the American College of Chest Physicians and the International Society for Heart and Lung Transplantation: endorsed by the Heart Rhythm Society. Circulation 2005; 112(12):e154-e235.
62. CONSENSUS Trial Study Group. Effects of enalapril on mortality in severe congestive heart failure. Results of the Cooperative North Scandinavian Enalapril Survival Study (CONSENSUS). N Engl J Med 1987; 316(23):1429-1435.
63. Pfeffer M A, Braunwald E, Moye L A, Basta L, Brown E J, Jr., Cuddy T E et al. Effect of captopril on mortality and morbidity in patients with left ventricular dysfunction after myocardial infarction. Results of the survival and ventricular enlargement trial. The SAVE Investigators. N Engl J Med 1992; 327(10):669-677.
64. SOLVD Investigators. Effect of enalapril on survival in patients with reduced left ventricular ejection fractions and congestive heart failure. N Engl J Med 1991; 325(5):293-302.
65. CIBIS-II Investigators and Committees. The Cardiac Insufficiency Bisoprolol Study II (CIBIS-II): a randomised trial. Lancet 1999; 353(9146):9-13.
66. Hjalmarson A, Goldstein S, Fagerberg B, Wedel H, Waagstein F, Kjekshus J et al. Effects of controlled-release metoprolol on total mortality, hospitalizations, and well-being in patients with heart failure: the Metoprolol CR/XL Randomized Intervention Trial in congestive heart failure (MERIT-HF). MERIT-HF Study Group. JAMA 2000; 283(10):1295-1302.
67. Packer M, Coats A J, Fowler M B, Katus H A, Krum H, Mohacsi P et al. Effect of carvedilol on survival in severe chronic heart failure. N Engl J Med 2001; 344(22):1651-1658.
68. Poole-Wilson P A, Swedberg K, Cleland J G, Di Lenarda A, Hanrath P, Komajda M et al. Comparison of carvedilol and metoprolol on clinical outcomes in patients with chronic heart failure in the Carvedilol Or Metoprolol European Trial (COMET): randomised controlled trial. Lancet 2003; 362(9377):7-13.
69. Pitt B, Zannad F, Remme W J, Cody R, Castaigne A, Perez A et al. The effect of spironolactone on morbidity and mortality in patients with severe heart failure. Randomized Aldactone Evaluation Study Investigators. N Engl J Med 1999; 341(10):709-717.
70. Pitt B, Remme W, Zannad F, Neaton J, Martinez F, Roniker B et al. Eplerenone, a selective aldosterone blocker, in patients with left ventricular dysfunction after myocardial infarction. N Engl J Med 2003; 348(14):1309-1321.
71. Pfeffer M A, McMurray J J, Velazquez E J, Rouleau J L, Kober L, Maggioni A P et al. Valsartan, captopril, or both in myocardial infarction complicated by heart failure, left ventricular dysfunction, or both. N Engl J Med 2003; 349(20):1893-1906.
72. McMurray J J, Ostergren J, Swedberg K, Granger C B, Held P, Michelson E L et al. Effects of candesartan in patients with chronic heart failure and reduced left-ventricular systolic function taking angiotensin-converting-enzyme inhibitors: the CHARM-Added trial. Lancet 2003; 362(9386):767-771.
73. Granger C B, McMurray J J, Yusuf S, Held P, Michelson E L, Olofsson B et al. Effects of candesartan in patients with chronic heart failure and reduced left-ventricular systolic function intolerant to angiotensin-converting-enzyme inhibitors: the CHARM-Alternative trial. Lancet 2003; 362(9386):772-776.
74. Bradley D J, Bradley E A, Baughman K L, Berger R D, Calkins H, Goodman S N et al. Cardiac resynchronization and death from progressive heart failure: a meta-analysis of randomized controlled trials. JAMA 2003; 289(6):730-740.
75. Bristow M R, Saxon L A, Boehmer J, Krueger S, Kass D A, De Marco T et al. Cardiac-resynchronization therapy with or without an implantable defibrillator in advanced chronic heart failure. N Engl J Med 2004; 350(21):2140-2150.
76. Cleland J G F, Daubert J C, Erdmann E, Freemantle N, Gras D, Kappenberger L et al. The Effect of Cardiac Resynchronization on Morbidity and Mortality in Heart Failure. N Engl J Med 2005; 352(15):1539-1549.
77. Moss A J, Zareba W, Hall W J, Klein H, Wilber D J, Cannom D S et al. Prophylactic implantation of a defibrillator in patients with myocardial infarction and reduced ejection fraction. N Engl J Med 2002; 346(12):877-883.
78. Kadish A, Dyer A, Daubert J P, Quigg R, Estes N A, Anderson K P et al. Prophylactic defibrillator implantation in patients with nonischemic dilated cardiomyopathy. N Engl J Med 2004; 350(21):2151-2158.
79. Bardy G H, Lee K L, Mark D B, Poole J E, Packer D L, Boineau R et al. Amiodarone or an implantable cardioverter-defibrillator for congestive heart failure. N Engl J Med 2005; 352(3):225-237.
80. Rose E A, Gelijns A C, Moskowitz A J, Heitjan D F, Stevenson L W, Dembitsky W et al. Long-term mechanical left ventricular assistance for end-stage heart failure. N Engl J Med 2001; 345(20):1435-1443.
81. Reynolds M R, Josephson M E. MADIT II (second Multicenter Automated Defibrillator Implantation Trial) debate: risk stratification, costs, and public policy. Circulation 2003; 108(15):1779-1783.
82. Cope J T, Kaza A K, Reade C C, Shockey K S, Kern J A, Tribble C G et al. A cost comparison of heart transplantation versus alternative operations for cardiomyopathy. Ann Thorac Surg 2001; 72(4):1298-1305.
83. Kittleson M M, Ye S Q, Irizarry R A, Minhas K M, Edness G, Conte J V et al. Identification of a gene expression profile that differentiates between ischemic and nonischemic cardiomyopathy. Circulation 2004; 110(22):3444-3451.

We claim:

1. A differential gene expression profile, comprising comparative gene expression levels resulting from gene expressions of a set of genes from patients having nonischemic cardiomyopathy compared to gene expressions of a set of corresponding genes from patients having nonfailing-hearts.

2. The differential gene expression profile of claim 1, wherein said set of genes are listed in Table 3.

3. The differential gene expression profile of claim 1, comprising Table 3.

4. A differential gene expression profile, comprising comparative gene expression levels resulting from gene expressions of a set of genes from patients having ischemic cardiomyopathy compared to gene expressions of a set of corresponding genes from patients having nonfailing-hearts.

5. The differential gene expression profile of claim 4, wherein said set of genes are listed in Table 4.

6. The differential gene expression profile of claim 4, comprising Table 4.

7. A gene expression profile for distinguishing between patients with left ventricular assist devices (LVADs) and without LVADs, comprising the genes listed in Table 6.
